(19) United States
(12) Patent Application Publication
(10) Pub. No.: US 2026/0027182 A1
(43) Pub. Date: Jan. 29, 2026

(54) MESENCEPHALIC ASTROCYTE-DERIVED NEUROTROPHIC FACTOR (MANF) FOR INCREASING MUSCLE REGENERATION

(71) Applicant: INSTITUTO DE MEDICINA MOLECULAR JOÃO LOBO ANTUNES, Lisboa (PT)

(72) Inventors: Pedro Sousa-Victor, Lisboa (PT); Joana Neves, Lisboa (PT); Neuza Sofia Simões De Sousa, Lisboa (PT)

(21) Appl. No.: 18/857,803

(22) PCT Filed: Mar. 23, 2023
(86) PCT No.: PCT/EP2023/057484

§ 371 Date: Oct. 17, 2024

(30) Foreign Application Priority Data
Apr. 21, 2022 (PT) 117925

(51) Int. Cl. A61K 38/18; A61P 21/00

(52) U.S. Cl. CPC A61K 38/18; A61P 21/00

(57) ABSTRACT

This invention relates to methods for increasing the regeneration of a skeletal muscle that comprise increasing the concentration of Mesencephalic Astrocyte-derived Neurotrophic Factor (MANF) in the skeletal muscle. The concentration of MANF may be increased by administering to the individual MANF, a nucleic acid encoding MANF, or a cell expressing MANF. This may be useful in the treatment of skeletal muscle damage or disease, for example in individuals with an age-related reduction in skeletal muscle regenerative capacity.

### CROSS-REFERENCE TO RELATED APPLICATIONS

This application is a U.S. National Stage Application of PCT Application No. PCT/EP2020/057484 filed Mar. 23, 2023, which application claims priority to Portuguese Application No. 117925 filed Apr. 21, 2022, the disclosure of which applications are incorporated herein by reference.

### REFERENCE TO AN ELECTRONIC SEQUENCE LISTING

The contents of the electronic sequence listing (MEWE-128_Sequence_Listing.xml; Size: 4,092 bytes; and Date of Creation: Oct. 2, 2025) is herein incorporated by reference in its entirety.

### FIELD

This invention relates to methods for increasing the regeneration of muscle in patients. This may be useful for example in treating muscle diseases and muscle damage.

### BACKGROUND

Age-related decline in regenerative capacity is the synergistic result of cell intrinsic impairments in somatic stem cells and alterations in the local and systemic environment coordinating the repair process^{1,2}. The reversible nature of some of the age-associated changes affecting the regenerative environment has been demonstrated through heterochronic parabiosis experiments, where old tissue is exposed to a youthful circulatory environment, highlighting the potential of interventions in the aged tissue milieu to reverse regenerative decline^{3,4}. The myeloid system is a potential candidate to mediate these rejuvenating effects⁵⁻⁷.

The skeletal muscle is a paradigmatic model to study age-related loss of regenerative capacity. Skeletal muscle regeneration is sustained throughout life by a population of adult muscle stem cells (MuSCs) and relies on a highly coordinated sequence of events, engaging several niche populations⁸⁻¹⁰. Immune cells infiltrate the skeletal muscle soon after injury and are responsible for essential functions, including the clearance of tissue debris and the coordinated regulation of MuSC function and other niche components^{11,12}. Macrophages are the most abundant type of immune cells participating in muscle repair and originate from infiltrating monocytes that differentiate in situ into pro-inflammatory macrophages¹². Regenerative success depends on a timely regulated phenotypic transition of these pro-inflammatory macrophages into pro-repair macrophages¹²⁻¹⁴. During this process CCR2^{pos}Ly6C^{High} macrophages mature into a different type of macrophage that loses Ly6C expression and acquires a new surface receptor, CX3CR1.

Recently, we identified a novel immune modulatory function for Mesencephalic Astrocyte-derived Neurotrophic Factor (MANF)¹⁵, an endoplasmic reticulum (ER)-stress-inducible protein with pleiotropic effects in multiple organs¹⁶⁻¹⁹. We found that MANF is a systemic regulator of inflammation and tissue homeostasis during ageing, and one of the factors required in young blood to promote part of the rejuvenation effects elicited by heterochronic parabiosis²⁰. Circulatory MANF levels decline with age in mice and humans, and MANF supplementation in old mice is sufficient to limit inflammation and tissue damage in the liver²⁰ and preserve retinal homeostasis²¹. However, how a decline in MANF signalling affects the age-related loss of regenerative capacity remains unexplored.

### SUMMARY

The present inventors have discovered that Mesencephalic Astrocyte-derived Neurotrophic Factor (MANF) increases the ability of skeletal muscle to regenerate and may be useful for example in treatment of individuals with skeletal muscle damage and/or skeletal muscle disease. Suitable individuals may include individuals with defects in muscle regeneration, for example age-related defects in muscle regeneration.

A first aspect of the invention provides a method of increasing the regeneration of a skeletal muscle in an individual in need thereof comprising;
increasing the concentration of Mesencephalic Astrocyte-derived Neurotrophic Factor (MANF) in the skeletal muscle.

The concentration of MANF may be increased by administering to the individual an agent that increases the concentration of MANF in the skeletal muscle.

A second aspect of the invention provides an agent that increases the concentration of MANF in a skeletal muscle for use in a method of increasing the regeneration of a skeletal muscle in an individual in need thereof.

A third aspect of the invention provides the use of an agent that increases the concentration of MANF in a muscle in the manufacture of a medicament for increasing the regeneration of a skeletal muscle in an individual in need thereof.

Suitable agents that increase the concentration of MANF for use in the first, second and third aspects may include (i) MANF (ii) a nucleic acid encoding MANF or (iii) a cell expressing MANF.

Skeletal muscle regeneration may be increased according to the first, second and third aspects for the treatment of skeletal muscle damage and/or disease in the individual.

An individual according to the first, second and third aspects may have a defect in skeletal muscle regeneration, for example, an age-related defect in skeletal muscle regeneration.

Other aspects and embodiments of the invention are described in more detail below.

### BRIEF DESCRIPTION OF THE FIGURES

FIG. 1 shows that MANF is essential for skeletal muscle regeneration. a, Western blot analysis of MANF levels in protein extracts of tibialis anterior (TA) muscles from yg wild type (wt, C57BL/6) mice, non-injured and at different time points following injury (2, 3, 4, 5, 6, 10 dpi). Ponceau S-staining of the membrane was used to verify equal protein loading in each sample. b, MANF protein levels, quantified by ELISA, in extracts of TA muscles from yg wt (C57BL/6) mice at different time points following injury (n=3-4/timepoint). c, Quantification of relative average levels of MANF, normalized to Ponceau levels, in protein extracts of TA muscles of yg (2-6 mo) and old (22-25 mo) wt (C57BL/6) mice at 3 dpi (n=3-6/age). d, Experimental timeline for analysis of animals with inducible and ubiquitous ablation of MANF. e, Representative images of eMHC (red) staining in cryosections of regenerating TA muscles from Manf^{fl/fl}, Manf^{R26WTa}nd Manf^{R26Δ} mice, at 4 dpi. DAPI is used to identify nuclei. Quantifications of this staining, for independent animals, are shown in FIG. 1f-h. f-h, Quantification of the average cross-sectional area of eMHC+ new myofibres (f), frequency distribution of new myofibres by size (g) and density of new myofibres (h) in regenerating TA muscles from Man^{fl/fl}, Manf^{R26WT} and Manf^{R26Δ} mice at 4 dpi (n=4-5/condition). i, Quantification of the number of necrotic fibres in regenerating TA muscles from Manf^{fl/fl}, Manf^{R26WT} and Manf^{R26Δ} mice at 4 dpi (n=4-5/condition). j-n, Quantification, by flow cytometry, of MuSCs (j), CD45^{pos}immune cells (k), myeloid cells (CD11bpos, l), pro-repair macrophages (F4/80^{pos}Ly6C^{Low}, m) and pro-inflammatory macrophages (Ly6C^{High}, n) in regenerating quadriceps (QC) muscles of Manf^{R26WT} and Manf^{R26Δ} mice at 3 dpi (j-k, n=3/condition; l-n, n=6-7/condition). o,q,r, Quantification, by flow cytometry, of myeloid cells (CD11b^{pos}, o), pro-repair macrophages (F4/80^{pos}Ly6C^{Low}, q) and pro-inflammatory macrophages (Ly6C^{High}, r) in regenerating QC muscles of yg (2-6 mo) and old (22-24 mo) wt (C57BL/6) mice at 3 dpi (n=4/age). p, Representative density plots from flow cytometry analysis of muscle cell populations from yg and old animals based on F4/80 and Ly6C cell surface markers, at 3 dpi. Percent numbers indicate pro-repair macrophages (F4/80^{pos}Ly6C^{Low}) relative to the CD11b^{pos}total population. Data are represented as average±s.e.m and each n represents one animal. In b, p values are from one-way ANOVA with Bonferroni's multiple comparison post-test. In all other graphs, p values are from two-tailed Student's t-test. yg, young; n.i., non-injured; dpi, days post-injury; i.p., intraperitoneal; eMHC; embryonic myosin heavy chain; CSA, cross-sectional area; MuSCs, muscle stem cells.

FIG. 2 shows that MANF derived from pro-repair macrophages is essential for skeletal muscle regeneration. a, Experimental timeline for analysis of animals treated with clodronate liposomes. b, Western blot analysis of MANF levels in protein extracts of QC muscles from wt (C57BL/6) mice, at 3 dpi, treated with clodronate liposomes (or control PBS liposomes). Quantification of relative average levels of MANF, normalized to Vinculin, is represented for each condition (n=6/condition). c, Western blot analysis of MANF levels in protein extracts from F4/80poscells FACS-isolated from QC muscles at different time points following injury. Quantification of relative average levels of MANF, normalized to Actin, is represented for each time-point (n=2, 2 and 3 dpi; n=3, 4 dpi; n=1 (pooled from 4 mice), 5 dpi). d, Western blot analysis of MANF levels in protein extracts from macrophage subpopulations (Ly6C^{High}and Ly6C^{Low}) FACS-isolated from QC muscles of wt mice, at 3 dpi. Actin was used to verify equal protein loading in each sample. e, Experimental timeline for analysis of animals with conditional ablation of MANF in Cx3cr1-expressing macrophages. f, Western blot analysis of MANF levels in protein extracts from F4/80poscells FACS-isolated from QC muscles of Manf^{Cx3cr1WT}and Manf^{Cx3cr1Δ} mice at 3 dpi. Actin was used to verify equal protein loading in each sample. g-i, Quantification, by flow cytometry, of myeloid cells (CD11bpos, g), pro-repair macrophages (F4/80^{pos}Ly6C^{Low}, h) and pro-inflammatory macrophages (Ly6C^{High}, i) in regenerating QC muscles of Manf^{Cx3cr1WT} and Manf^{Cx3cr1Δ} mice at different time points following injury (n=6-11/condition). j, Ratio of pro-repair to pro-inflammatory macrophages in regenerating QC muscles of Manf^{Cx3cr1WT} and Manf^{Cx3cr1Δ} at 3 dpi (n=9-11/condition). k. Quantification by flow cytometry of MUSCs in regenerating QC muscles of Manf^{Cx3cr1WT} and Manf^{Cx3cr1Δ} mice at 3 dpi (n=9-11/condition). l, Representative images of eMHC (red) staining in cryosections of regenerating TA muscle from Manf^{Cx3cr1WT}and Manf^{Cx3cr1Δ} mice, at 4 dpi. DAPI is used to identify nuclei. Quantifications of this staining, for independent animals, are shown in FIG. 2m-n. m-n, Quantification of the average cross-sectional area of eMHC+ new myofibres (m) and frequency distribution of new myofibres by size (n), in regenerating TA muscles from Manf^{Cx3cr1WT} and Manf^{Cx3cr1Δ} mice at 4 dpi (n=6/condition). o, Quantification of the number of necrotic fibres in regenerating TA muscles from Manf^{Cx3cr1WT} and Manf^{Cx3cr1Δ} mice at 4 dpi (n=8/condition). Data are represented as average±s.e.m. and each n represents one animal. p values are from two-tailed Student's t-test. dpi, days post-injury; i.v., intravenous injection; PBS-Lipo, PBS Liposomes; Clo-Lipo, Clodronate Liposomes; FACS, Fluorescence activated cell sorting; i.p., intraperitoneal injection; eMHC; embryonic myosin heavy chain; CSA, cross-sectional area; MuSCs, muscle stem cells.

FIG. 3 shows that MANF-deficient macrophages have a delayed phenotypic transition and altered structural properties a, Percentage of pro-inflammatory (Ly6C^{High}) macrophages, quantified by flow cytometry, in single cells suspensions isolated from QC muscles of Man^{fl/fl} and Manf^{LysMΔ} mice at 2 dpi, 0 h and 16 h after culture, with or without recombinant MANF supplementation (n=10-15/condition). Example of the gating strategy to define the monocyte/macrophage population is shown in Extended data 4f, and representative histograms of the Ly6C signal in the selected population is shown in Extended data 4 g at 0 h and 16 h, and the Ly6C^{High}gate is defined. b, MFI in the Ly6C-FITC channel, quantified by flow cytometry, in Ly6C^{High}macrophages FACSorted from QC muscles of Manf^{fl/fl} and Manf^{LysMΔ} mice at 2 dpi, 0 h and 16 h after culture (n=6/condition). Representative histogram of the Ly6C signal in the selected population is shown in Extended data 4 h at 0 h and 16 h, for cells FACSorted from Man^{fl/fl} and Manf^{LysMΔ} mice. c, Relative levels of Manf and Cx3cr1 mRNA, detected by RT-qPCR, in Ly6CHighmacrophages FACSorted from QC muscles of Manffl/fl and Manf^{R26Δ} mice at 2 dpi, 0 h and 20 h after culture (n=3-6/condition). d,e, GO categories of cellular components (d) and molecular functions (e) showing significant enrichment in the dataset of genes down-regulated in macrophages (CD45^{pos}F4/80^{pos}) FACS-isolated at 3 dpi from QC muscles of Manf^{Cx3cr1Δ} mice compared to Manf^{Cx3cr1WT} mice (fold change<0.75 and p≤0.05, p values from two-tailed Student's t-test, n=3/condition). f-h, Quantification of macrophage area (f), intracellular vesicle area (g), and percentage of pro-repair macrophages with at least one vesicle bigger than 5 sqμm (h), in a population of pro-repair macrophages FACS-isolated at 3 dpi from QC muscles of Manf^{Cx3cr1WT} and Manf^{Cx3cr1Δ} mice (n=54 macrophages, Manf^{Cx3cr1WT} and n=42 macrophages, Manf^{Cx3cr1Δ}). i, MFI of Fluoresbrite 641 signal, quantified by flow cytometry, in Manf^{WT} and Manf^{KO} BMDMs, 3 h after stimulation with opsonized Fluoresbrite® 641 Carboxylate beads (n=4-5/condition). Representative histogram of the Fluoresbrite® 641 signal is shown in Extended data 5b. j, Lysosomal activity, evaluated by flow cytometry, as the MFI in the FITC channel, in Manf^{WT} and Manf^{KO}BMDMs (n=3/condition), 3 h after stimulation with a self-quenched lysosomal substrate and cell debris containing apoptotic and necrotic cells (Apop-Necro). Lysosomal activity is referred to the basal lysosomal activity in each individual BMDM culture. Data are represented as average±s.e.m. and each n represents one animal unless otherwise specified. In c, p values are from one-way ANOVA with Bonferroni's multiple comparison post-test. For all other graphs, p values are from two-tailed Student's t-test. dpi, days post-injury; FC, flow cytometry; rMANF; recombinant Mesencephalic astrocyte-derived neurotrophic factor; GO, Gene Ontology; CC, cellular component; MF, molecular function; MFI, Mean Fluorescence intensity; Baf, Bafilomycin.

FIG. 4 shows that MANF therapy restores the repair-associated myeloid response and regenerative success in aged skeletal muscles. a,b, GO categories of biological processes (a) and cellular components (b) showing significant enrichment in the dataset of genes up-regulated (a) or down-regulated (b) in pro-repair macrophages (F4/80posLy6C^{Low}) FACS-isolated at 3 dpi from QC muscles of old (22-24 mo) mice compared to yg (2-6 mo) mice (fold change<0.75 or >1.5 and p≤0.05, p values from two-tailed Student's t-test, n=3/condition). c, Lysosomal activity, evaluated by flow cytometry, as the MFI in the FITC channel, in BMDMs from yg and old mice, with and without rMANF stimulation (n=3-7/condition), 3 h after stimulation with a self-quenched lysosomal substrate and cell debris containing apoptotic and necrotic cells (Apop-Necro). Lysosomal activity is referred to the basal lysosomal activity in each individual BMDM culture. Representative histograms of the FITC signal derived from lysosomal hydrolysis of the self-quenched substrate in basal conditions and after stimulation with Apop-necro is shown in Extended data 5e for all condition. d, Experimental timeline for analysis of aged animals after receiving intramuscular injections of rMANF protein. e-g, Quantification, by flow cytometry, of myeloid cells (CD11bpos, d), pro-repair macrophages (F4/80posLy6CLow+Int, e) and pro-inflammatory macrophages (Ly6C^{High}, f) in regenerating TA muscles of yg (2-3 mo) and old (23-25 mo) wt (C57BL/6) mice at 3 dpi, treated with i.m. injections of saline or rMANF (n=3-8/condition). h, Representative images of eMHC (red, left) or mouse IgG (right) staining in cryosections of regenerating TA muscles from old (25 mo) mice, at 4 dpi, treated with intramuscular injections of rMANF or saline. DAPI (blue) is used to identify nuclei. Asterisks indicate necrotic myofibres. Quantifications of these stainings, for independent animals, are shown in FIG. 4h-j. i-k, Quantification of the average cross-sectional area of new myofibers (i), frequency distribution of new myofibres by size (j) and average number of necrotic fibres (k) in regenerating TA muscles from old (25 mo) mice, at 4 dpi, treated with intramuscular injections of rMANF or saline (n=3/condition). Data are represented as average±s.e.m. and each n represents one animal. In c, e-g, p values are from one-way ANOVA with Bonferroni's multiple comparison post-test. In i, k, p values are from two-tailed Student's t-test. GO, Gene Ontology; BP, biological process; CC, cellular component; i.m., intramuscular injection; rMANF; recombinant Mesencephalic astrocyte-derived neurotrophic factor; eMHC; embryonic myosin heavy chain; ms IgG, mouse Immunoglobulin; CSA, cross-sectional area

### DETAILED DESCRIPTION

This invention relates to the finding that the capacity of skeletal muscle to regenerate following damage is increased by Mesencephalic Astrocyte-derived Neurotrophic Factor (MANF). The administration of MANF may therefore be useful in promoting or improving skeletal muscle regeneration, for example in the treatment of skeletal muscle damage or disease. In some embodiments, individuals suitable for treatment may include individuals with defective or impaired muscle regeneration, for example age-related defects or impairments in muscle regeneration.

MANF as described herein may increase or restore the repair-associated myeloid response and/or increase or restore the numbers of myeloid cells and pro-repair macrophages in a damaged skeletal muscle. This may increase or promote the repair and regeneration of the skeletal muscle after damage.

The capacity of the skeletal muscle to regenerate after damage may be increased by MANF as described herein. For example, the density and cross-sectional area (CSA) of new myofibres may be increased in the skeletal muscle and the accumulation of uncleared necrotic fibres may be reduced.

The concentration of MANF in a skeletal muscle may be increased relative to the concentration before treatment by an amount sufficient to positively affect the regenerative capacity of the skeletal muscle. For example, the concentration of MANF in a skeletal muscle may be increased by at least 10% over the concentration of MANF in the skeletal muscle prior to treatment, e.g. 20% or more, 30% or more, 40% or more, 50% or more, 100% or more, 150% or more, 200% or more, 250% or more, 300% or more, or 350% or more.

Mesencephalic Astrocyte-derived Neurotrophic Factor (MANF) is an 18 kDa endoplasmic reticulum (ER)-stress-inducible protein that has pleiotropic effects in various organs. MANF as described herein may be human MANF. Human MANF (also known as ARP or ARMET; Gene ID 7873) has the reference amino acid sequence of NCBI database entry NP_006001.5 (SEQ ID NO:1) and is encoded by the reference nucleotide sequence of NCBI database entry NM_006010.6 (SEQ ID NO: 2).

To increase the regeneration of a skeletal muscle as described herein a method may comprise administering to an individual an effective amount of an agent that increases the concentration of MANF in skeletal muscle.

Preferably, the agent is a MANF. A method described herein may comprise administering a MANF to an individual in order to increase the regeneration of skeletal muscle in the individual.

A MANF may comprise the amino acid sequence of residues 25 to 182 of SEQ ID NO: 1 or may be a variant thereof. A variant of a reference amino acid sequence set out herein may comprise an amino acid sequence or a nucleotide sequence having at least 50%, at least 60%, at least 70%, at least 80%, at least 90%, at least 95%, or at least 98% sequence identity to the reference sequence. Particular amino acid sequence variants may differ from the reference sequence by insertion, addition, substitution or deletion of 1 amino acid, 2, 3, 4, 5, 6, 7, 8, 9, or 10 or more than 10 amino acids. For example, a MANF may have 50 or fewer amino acid residues altered relative to a the mature human MANF sequence of residues 25 to 182 of SEQ ID NO 1, preferably 45 or fewer, 40 or fewer, 30 or fewer, 20 or fewer, 15 or fewer, 10 or fewer, 5 or fewer or 3 or fewer.

Sequence similarity and identity are commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85:2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm (Nucl. Acids Res. (1997) 25 3389-3402) may be used. Computerized implementations of these algorithms (GAP, BESTFIT, PASTA, and FASTA in the Wisconsin Genetics Software Package, Genetics Computer Group, 575 Science Dr., Madison, WI) are available and publicly available computer software may be used such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)), Genomequest™ software (Gene-IT, Worcester MA USA) and MAFFT (Katoh and Standley 2013, Molecular Biology and Evolution, 30 (4) 772-780 software. When using such software, the default parameters, e.g. for gap penalty and extension penalty, are preferably used. A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Sequence comparison may be made over the full-length of the relevant sequence described herein.

An amino acid residue in a reference amino acid sequence may be altered or mutated by insertion, deletion or substitution, preferably substitution for a different amino acid residue, to produce a variant of the reference amino acid sequence. Such alterations may be caused by one or more of addition, insertion, deletion or substitution of one or more nucleotides in the encoding nucleic acid. A MANF may, for example, comprise an amino acid sequence which differs from the sequence of residues 25 to 182 of SEQ ID NO: 1 by insertion, addition, substitution or deletion of 1 amino acid, 2, 3, 4, 5-10, 10-20 or 20-30 amino acids.

In some embodiments, one or more amino acid residues in the MANF may be non-natural amino acids, modified amino acids or D-amino acids. The use of such amino acids is well-known to those of skill in the art

In some embodiments, a MANF as described herein may be part of a fusion protein which contains one or more heterologous amino acid sequences additional to the MANF sequence. For example, the fusion protein comprising the MANF may further comprise one or more additional domains which improve the stability, pharmacokinetic, targeting, affinity, purification and production properties of the MANF. Suitable additional domains include immunoglobulin Fc domains. Immunoglobulin Fc domains are well-known in the art and include the human IgG1 Fc domain. A human immunoglobulin Fc domain may be located at the N-terminal or C-terminal end of the MANF.

MANF may be produced using synthetic or recombinant techniques which are standard in the art. For example, MANF may be generated wholly or partly by chemical synthesis. Suitable techniques include liquid or solid-phase synthesis methods; in solution; or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof. The chemical synthesis of polypeptides is well-known in the art (J. M. Stewart and J. D. Young, Solid Phase Peptide Synthesis, 2nd edition, Pierce Chemical Company, Rockford, Illinois (1984); M. Bodanzsky and A. Bodanzsky, The Practice of Peptide Synthesis, Springer Verlag, New York (1984); J. H. Jones, The Chemical Synthesis of Peptides. Oxford University Press, Oxford 1991; in Applied Biosystems 430A Users Manual, ABI Inc., Foster City, California; G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997).

Alternatively, MANF may be generated wholly or partly by recombinant techniques. For example, a nucleic acid encoding MANF may be expressed in a host cell and the expressed polypeptide isolated and/or purified from the cell culture. For the production of recombinant MANF, nucleic acid sequences encoding MANF may be comprised within an expression vector. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the MANF nucleic acid in a host cell. Suitable regulatory sequences to drive the expression of heterologous nucleic acid coding sequences in a range of expression systems are well-known in the art and include constitutive promoters, for example viral promoters such as CMV or SV40, and inducible promoters, such as Tet-on controlled promoters. A vector may also comprise sequences, such as origins of replication and selectable markers, which allow for its selection and replication and expression in suitable bacterial hosts and/or in eukaryotic cells, such as yeast, insect or mammalian cells. Vectors suitable for use in expressing MANF nucleic acid include plasmids and viral vectors e.g. ‘phage, or phagemid, and the precise choice of vector will depend on the particular expression system which is employed. MANF may be expressed in any convenient expression system, and numerous suitable systems are available in the art, including bacterial, yeast, insect or mammalian cell expression systems. For further details see, for example, Molecular Cloning: a Laboratory Manual: 3rd edition, Russell et al., 2001, Cold Spring Harbor Laboratory Press. Techniques and protocols for expression of recombinant polypeptides in cell culture and their subsequent isolation and purification are well known in the art (see for example Protocols in Molecular Biology, Second Edition, Ausubel et al. eds. John Wiley & Sons, 1992; Recombinant Gene Expression Protocols Ed RS Tuan (March 1997) Humana Press Inc). The expressed polypeptide comprising or consisting of the MANF may be isolated and/or purified, after production. This may be achieved using any convenient method known in the art. Techniques for the purification of recombinant polypeptides are well known in the art and include, for example HPLC, FPLC or affinity chromatography.

In other embodiments, the agent that increases the concentration of MANF in skeletal muscle may be a nucleic acid that encodes a MANF. Following administration, the expression of the nucleic acid in the skeletal muscle may increase the concentration of MANF in the muscle. A method described herein may comprise administering a nucleic acid that encodes a MANF to the individual in order to increase the regeneration of skeletal muscle in the individual.

A nucleic acid encoding MANF may comprise the nucleotide sequence of SEQ ID NO: 2 or a variant thereof.

A variant of a reference nucleotide sequence set out herein may comprise a nucleotide sequence having at least 50%, at least 60%, at least 70%, at least 80%, at least 90%, at least 95%, or at least 98% sequence identity to the reference sequence. Particular nucleotide sequence variants may differ from the reference sequence by insertion, addition, substitution or deletion of 1 nucleotide, 2, 3, 4, 5, 6, 7, 8, 9, or 10 or more than 10 nucleotides. A nucleotide in a reference nucleotide sequence may be altered or mutated by insertion, deletion or substitution, preferably substitution for a different nucleotide, to produce a variant of the reference nucleotide sequence.

The nucleic acid encoding MANF may be operably linked to a regulatory element, such that the nucleic acid is expressed in the individual, preferably in a skeletal muscle of the individual. Suitable regulatory elements are well known in the art. Preferably, the nucleic acid encoding MANF is contained in a vector. Vectors suitable for administration to an individual e.g. for gene therapy applications are well known in the art. For example, suitable vectors may include viral vectors, such as retroviral vectors, lentiviral vectors, adenoviral vectors and adenovirus-associated virus (AAV) vectors.

In other embodiments, the agent that increases the concentration of MANF in skeletal muscle may be a cell that expresses MANF. Following administration, the expression of MANF by the cell may increase the concentration of MANF in the skeletal muscle. A method described herein may comprise administering a cell that expresses MANF to the individual in order to increase the regeneration of skeletal muscle in the individual.

The cell may comprise a heterologous nucleic acid that encodes MANF. Nucleic acid that encodes MANF is described above. Suitable cells include mammalian cells, preferably human cells, including immune cells, such as macrophages, muscle cells, such as myocytes, myoblasts, and muscle stem cells; and stromal cells, such as fibroblasts, fibro adipogenic progenitors and other mesenchymal stem cells.

In other embodiments, the agent that increases the concentration of MANF in skeletal muscle may be a small organic molecule, for example an organic compound having a molecular weight of 900 Da or less. A suitable compound may directly or indirectly increase the concentration of MANF in skeletal muscle. Suitable compounds may for example include tunicamycin, thapsigargin, lactatystin and analogues, variants and derivatives thereof (Mizobuchi et al Cell Struct Funct 2007 32:41-50; Apostolou et al Exp Cell Res 2008 314 2454-2467; Kim et al Tranls Res 2017 188 1-9).

An agent that increases the concentration of MANF, for example MANF, a nucleic that encodes MANF or a cell that expresses MANF, may be administered to an individual as described herein to promote skeletal muscle regeneration or increase the capacity of skeletal muscle to regenerate. While it is possible for the agent to be administered alone in the methods described herein, it is preferable to present it as a pharmaceutical composition (e.g. a formulation) comprising the agent together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents. Pharmaceutical compositions comprising an agent that increases the concentration of MANF as described above admixed or formulated together with one or more pharmaceutically acceptable carriers, excipients, buffers, adjuvants, stabilisers, or other materials, as described herein, may be used in the methods described above.

The term “pharmaceutically acceptable” relates to compounds, materials, compositions, and/or dosage forms which are, within the scope of sound veterinary or medical judgement, suitable for use in contact with the tissues of a subject (e.g. human or other mammal) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable excipients and carriers include, without limitation, water, saline, buffered saline, phosphate buffer, alcoholic/aqueous solutions, emulsions or suspensions. Other conventionally employed diluents, adjuvants, and excipients may be added in accordance with conventional techniques. Such carriers can include ethanol, polyols, and suitable mixtures thereof, vegetable oils, and injectable organic esters. Buffers and pH-adjusting agents may also be employed, and include, without limitation, salts prepared from an organic acid or base. Representative buffers include, without limitation, organic acid salts, such as salts of citric acid (e.g., citrates), ascorbic acid, gluconic acid, carbonic acid, tartaric acid, succinic acid, acetic acid, phthalic acid, Tris, trimethylamine hydrochloride, or phosphate buffers. Parenteral carriers can include sodium chloride solution, Ringer's dextrose, dextrose, trehalose, sucrose, lactated Ringer's, or fixed oils. Intravenous carriers can include fluid and nutrient replenishers, electrolyte replenishers, such as those based on Ringer's dextrose, and the like. Preservatives and other additives such as, for example, antimicrobials, antioxidants, chelating agents (e.g., EGTA; EDTA), inert gases, and the like may also be provided in the pharmaceutical carriers. The pharmaceutical compositions described herein are not limited by the selection of the carrier. The preparation of these pharmaceutically acceptable compositions, from the above-described components, having appropriate pH, isotonicity, stability and other conventional characteristics, is within the skill of the art. Suitable carriers, excipients, etc. may be found in standard pharmaceutical texts, for example, Remington's Pharmaceutical Sciences and The Handbook of Pharmaceutical Excipients, 4th edit, eds. R. C. Rowe et al, APhA Publications, 2003.

Pharmaceutical compositions may conveniently be presented in unit dosage form and may be prepared by any methods well-known in the art of pharmacy. Such methods include the step of bringing the one or more isolated conjugates/immunogenic polypeptides into association with a carrier or excipient as described above which may constitute one or more accessory ingredients. In general, the formulations are prepared by uniformly and intimately bringing into association the active compound with liquid carriers or finely divided solid carriers or both.

Pharmaceutical compositions may be made in the form of sterile aqueous solutions or dispersions, suitable for injectable use, or made in lyophilized forms using freeze-drying techniques. Lyophilized pharmaceutical compositions are typically maintained at about 4° C., and can be reconstituted in a stabilizing solution, e.g., saline or HEPES, with or without adjuvant. Pharmaceutical compositions may be presented in unit-dose or multi-dose sealed containers, for example, ampoules and vials, and may be stored in a freeze-dried (lyophilised) condition requiring only the addition of the sterile liquid carrier, for example water for injections immediately prior to use.

An agent described above; or a pharmaceutical composition comprising the agent may be administered to a subject by any convenient route of administration.

In some preferred embodiments, the agent may be administered directly to a skeletal muscle, for example by intra-muscular administration, preferably intra-muscular injection (IM). For example, the agent may be administered to a site of damage or impaired regeneration in the muscle, such that the concentration of MANF is increased at the site.

Formulations suitable for intramuscular injection are well known in the art and include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Typically, the concentration of the active compound in the solution is from about 1 μg/ml to about 100 mg/ml, for example, from about 10 μg/ml to about 50 mg/ml.

In other embodiments, the agent may be administered systemically to the individual. This may increase circulatory levels of MANF in the individual and consequently lead to an increase in the concentration of MANF in skeletal muscle. but not limited to; parenteral, for example, by injection, including intravenous, sub-cutaneous or intraperitoneal injection. Suitable techniques are known in the art and commonly used in therapy.

Preferably, an agent as described herein are formulated in a pharmaceutical composition for intra-muscular, intra-venous or sub-cutaneous administration.

It will be appreciated that appropriate dosages of the agent can vary from patient to patient, depending on the circumstances. Determining the optimal dosage will generally involve the balancing of the level of therapeutic benefit against any risk or deleterious side effects of the administration. The selected dosage level will depend on a variety of factors including, but not limited to, the route of administration, the time of administration, the in vivo half-life of the agent, rate of depletion of MANF concentration in the muscle, other drugs, compounds, and/or materials used in combination, and the maturity, sex, weight, condition and general health of the patient. The amount of agent and route of administration will ultimately be at the discretion of the physician, although generally the dosage will be to achieve concentrations of MANF in the skeletal muscle which are sufficient to produce a beneficial effect without causing substantial harmful or deleterious side-effects.

Administration in vivo can be effected in one dose, continuously or intermittently (e.g., in divided doses at appropriate intervals). Methods of determining the most effective means and dosage of administration are well known to those of skill in the art and will vary with the formulation and the subject being treated. Single or multiple administrations may be carried out with the dose level and pattern being selected by the physician.

Administration is normally in a “therapeutically effective amount” or “prophylactically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners.

A composition may be administered alone or in combination with other treatments, either simultaneously or sequentially dependent upon the circumstances of the individual to be treated.

Prescription of treatment, e.g. decisions on dosage etc, is within the responsibility of general practitioners and other medical doctors and may depend on the severity of the symptoms and/or progression of a disease being treated. Appropriate doses of therapeutic polypeptides and other agents are well known in the art (Ledermann J. A. et al. (1991) Int. J. Cancer 47:659-664; Bagshawe K. D. et al. (1991) Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an agent may be determined by comparing its in vitro activity and in vivo activity in an animal model. Methods for extrapolation of effective dosages in mice and other test animals to humans are known. The precise dose will depend upon a number of factors, including whether the agent is for prevention or for treatment, the size and location of the area to be treated, the precise nature of the agent and the nature of any detectable label or other molecule attached to the agent.

A typical dose of MANF may for example increase the concentration of MANF at the site of injury to 3 or more, 4 or more, 5 or more or 6 or more ng/ml, for example 3-6 ng/ml. In some embodiments, the concentration of MANF at the site of injury may be increased to 3 or more, 4 or more, 5 or more or 6 or more ng/ml, for example 3-6 ng/ml. An initial higher loading dose, followed by one or more lower doses, may be administered. Treatment may be periodic, and the period between administrations may be about one week or more, e.g. about two weeks or more, about three weeks or more, about four weeks or more, about once a month or more, about five weeks or more, or about six weeks or more. For example, treatment may be every two to four weeks or every four to eight weeks. For example, treatments may be repeated at daily, twice-weekly, weekly or monthly intervals, at the discretion of the physician. The treatment schedule for an individual may be dependent on the individual, the pharmacokinetic and pharmacodynamic properties of the agent, the route of administration and the nature of the condition being treated, for example the time and nature of a muscle trauma. MANF may for example be administered for 1, 2, 3, 4, 5, 6, or 7 days following muscle trauma, or for longer periods in the case of muscle disease.

The term “treatment”, as used herein in the context of treating a condition, pertains generally to treatment and therapy in which some desired therapeutic effect is achieved, for example, the inhibition of the progress of the condition, and includes a reduction in the rate of progress, a halt in the rate of progress and amelioration of the condition, and cure of the condition.

The methods described herein increase or promote skeletal muscle regeneration in an individual and/or increase or restore the regenerative capacity of skeletal muscle in an individual.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

Preferably, the individual is a human. However, non-human mammals, especially mammals that are conventionally used as models for demonstrating therapeutic efficacy in humans (e.g. murine, primate, porcine, canine, or leporine animals) may also be employed.

In some embodiments, an individual suitable for treatment as described herein may have reduced capacity for muscle regeneration. For example, the individual may have a defect in muscle regeneration. Defects in muscle regeneration may include reduced formation of new myofibres; formation of new myofibres with reduced density; formation of new myofibres with reduced cross-sectional area (iii) reduced clearance of necrotic myofibres (iv) reduced capacity of a muscle to produce force (muscle function) (v) increased fibrosis and/or (vi) increased fatty degeneration relative to healthy controls following muscle damage. Healthy controls may include individuals with normal muscle regeneration capacity, for example individuals without defects in muscle regeneration.

In some embodiments, one or more of the formation of new myofibres; the formation of new myofibres with reduced density or cross-sectional area; clearance of necrotic myofibres; capacity of a muscle to produce force (muscle function); fibrosis; and fatty degeneration may be used as markers to assess evaluating regeneration of skeletal muscle following treatment as described herein.

Loss of regenerative capacity in skeletal muscle is an established feature of aging. Defects in muscle regeneration may be age-related defects. Age-related defects are defects that are caused by or arise from the aging process. For example, an individual suitable for treatment as described herein may be mature or elderly. The age of the individual may be 60% or more, 65% or more, 70% or more, 75% or more or 80% or more of the average life expectancy of the individual's population group. In some embodiments, the individual may be a human of 50 years or older, 55 years or older, 60 years or older, 65 years or older or 70 years or older. Age-related defects in muscle regeneration may be characterised by a reduced capacity or an incapacity to regain muscle function relative to healthy controls following muscle damage.

An individual suitable for treatment as described herein may have skeletal muscle damage. Methods described herein may promote or increase the recovery or repair of the skeletal muscle damage in the individual. In some embodiments, muscle damage may be caused by traumatic injury or exertion.

Muscle damage may for example include trauma related to falls or accidents, muscle damage occurring during surgery (patients undergoing surgical interventions such as tumour ablation, soft tissue reconstruction, or joint arthroplasty), bruising, spraining or lacerations connected with exercise or strong exertion, or misuse of muscle groups.

An individual suitable for treatment as described herein may have a muscle disease. For example, the individual may have a muscle disease. Muscle diseases may include sarcopenia; muscular dystrophy, such as Duchenne Muscle Dystrophy; neuromuscular diseases, such as spinal muscular atrophy, peripheral nerve diseases, Amyotrophic Lateral Sclerosis (ALS), and neuromuscular junction disease; and myopathies, such as inflammatory myopathy, dermatomyositis, mitochondrial myopathy and metabolic myopathy.

An individual suitable for treatment as described herein may have reduced muscle mass and/or function following immobilization, for example during post-surgical hospitalization.

In some embodiments, a method described herein may further comprise administering a second therapy, such as exercise therapy, to the individual; or a second therapeutic agent to the individual. Second therapeutic agents may include immune cells, such as macrophages.

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

Other aspects and embodiments of the invention provide the aspects and embodiments described above with the term “comprising” replaced by the term “consisting of” and the aspects and embodiments described above with the term “comprising” replaced by the term “consisting essentially of”.

The term “downstream” as used herein refers to the 5′ to 3′ direction in a nucleic acid described herein and the term “upstream” as used herein refers to the 3′ to 5′ direction in a nucleic acid described herein

Reference to a nucleotide sequence as set out herein encompasses a DNA molecule with the specified sequence, and encompasses a RNA molecule with the specified sequence in which U is substituted for T, unless context requires otherwise.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

### EXPERIMENTAL

### Methods

### Animals

All mice used in these studies were housed at the DGAV accredited rodent facility of Instituto de Medicina Molecular, in individually ventilated cages within a specific and opportunistic pathogen-free (SPOF) facility, on a standard 12/12 h light cycle. The care and use of experimental animals complied with relevant institutional and national animal welfare laws, guidelines and policies. Old wt C57BL/6 mice were purchased from Charles River, Europe with 18-20 months and further aged in house until analysis. Young wt mice were either purchased from Charles River, Europe; or born in house and generated using C57BL/6 breeders purchased from Charles River, Europe. To create a mouse model allowing an inducible and ubiquitous ablation of Manf we generated Rosa26CRE-ERT/+Manf^{fl/fl} mice. Mice carrying the Rosa26CRE-ERTallele are B6; 129-Gt (ROSA) 26Sortm1 (cre/ERT) Nat/J and were purchased from The Jackson Laboratory (JAX, stock no: 004847). In these mice a Cre^{ERT} cassette is inserted within intron 1 of the GT (ROSA) 26Sor locus and is expressed under the control of its endogenous promoter. Mice carrying the Manf^{fl}allele were previously described²⁰. Heterozygous carriers of the Cre^{ERT}allele and homozygous carriers of the Manf^{fl}allele were used for these studies. Cx3cr1^{CRE-ER/+}. Man^{fl/fl}mice were previously described²⁰. To induce Cre activity, mice received daily intra-peritoneal injections of tamoxifen (T5648-1G-Sigma) in sterile corn oil, at a dose of 75 mg/Kg of body weight. Control mice were littermates of the same genotype that received sham injections (corn oil) or Manf^{fl/fl} mice that received tamoxifen injections. To create a mouse model where MANF is ablated in macrophages in a tamoxifen-independent manner we generated LysM^{CRE/+/}MANF^{fl/fl} mice. Mice carrying the LysM^{CRE} allele are B6.129P2-Lyz2tm1(cre)lfo/J and were purchased from JAX (stock number 004781). Heterozygous carriers of the Cre allele and homozygous carriers of the Manf^{fl} allele were used for these studies. Control mice were MANF^{fl/fl} littermates without the Cre allele. LysM^{CRE/+}, Manf^{fl/fl}; Cx3cr1^{CRE-ER/+}. Manf^{fl/fl} mice; and MANF^{fl/fl} mice were generated at the Buck Institute for Research on Aging (Novato, CA, USA) and re-derived into SPOF C57BL/6J strain by in vitro fertilization.

### In Vivo Procedures in Mice

All procedures involving animals were approved by Direção Geral da Alimentação e Veterinaria (DGAV) and performed at the rodent facility of Instituto de Medicina Molecular.

Induction of muscle regeneration: Regeneration of skeletal muscle was induced by intramuscular (i.m.) injection of sterile 1.2% Barium Chloride (Sigma: 342920) in saline solution (0.9% NaCl, B. BRAUN) into the tibialis anterior (TA, 40 ul) or quadriceps (QC, 50 ul) muscle of the mice. At the designated time points after injury, mice were euthanized and muscles were collected for analysis. Animals were anesthetized for the procedure with Isoflurane inhalation. Tamoxifen injected mice were anesthetized with Ketamine 75 mg/Kg of body weight+Medetomidine 1 mg/Kg of body weight before i.m.injection.

Macrophage ablation: Chemical ablation of macrophages was performed using a clodronate-liposome solution. Clodronate-liposomes or PBS-liposomes (LIPOSOMA—Research solution SKU: CP-010-010) at 5 mg/ml were inject intravenously (tail) at a dose of 100 ul/10 g of body weight. Animals received one injection on the day before injury, and then daily injections until analysis starting at 1 dpi.

rMANF intramuscular injection: Injured mice received daily i.m. injections of 20 ul of saline solution (0.9% NaCl, B. BRAUN) containing 2 μg or 4 μg of hrMANF protein (P-101-100, Icosagen), in the injured muscle starting at 1 dpi until the day of analysis. Control animals in the same experiment received the same regimen of i.m. injections of saline solution without rMANF supplementation. hrMANF protein used had less than 1EU of endotoxin per mg of protein.

In Vivo EdU (5-ethynyl-2′-deoxyuridine) Labeling: EdU labelling was performed by intraperitoneal injection of 200 ul of EdU (3 mg/ml) dissolved in PBS, 24 h prior to analysis.

### Histological Analysis, Imaging and Quantification Methods for Muscle Tissue

Muscle tissue harvesting and storage: Animals were euthanized and tissues were harvested. For histology analysis the dissected TA muscle was mounted vertically on a tragacanth gum (G1128-100G—Sigma) placed on a cardboard. The tissue was frozen in methyl-butane (VWRC103614T—VWR) cooled with liquid nitrogen, for about 13 to 17 seconds depending on muscle size and stored at −80° C. for further analysis as described60. Frozen tissue was cryosectioned at 10 μm thickness on a cryostat (LEICA CM 3050S), collected on Superfrost microslides (VWR) and stored at −80° C. until analysis. Samples processed for RNA and protein analysis were flash frozen in cryotubes submerged in liquid nitrogen.

H&E staining of muscle sections: Muscle cryosections collected on slides were thawed at room temperature (RT) for 10 min. Cryosections were placed in distilled water for 5 min, stained on Harris Hematoxylin (05-06004E-Enzifarma), placed under running water for 5 min, dipped on ethanol 70%, stained on eosin (HT110132-1L—Sigma). Stained tissue was serially dehydrated in 70%, 95% and 100% (twice) ethanol for 30 sec on each alcohol, incubated in xylene (3803665EGDG—Leica Microsystems) for at least 10 min and mounted with MICROMOUNT (3801731DG-Leica Microsystems).

Immunohistochemistry (IHC) of muscle cryosections and nuclei staining: Muscle cryosections collected on slides were thawed, permeabilized with PFA 4% in Phosphate-buffered saline (PBS) 10 min at RT, incubated in boiling 10 mM Citrate buffer 45 min, blocked with Mouse on Mouse Blocking Reagent (R&D Systems) 2 h and incubated with primary antibody, diluted in blocking solution, overnight (O/N) at 4° C. Primary antibody was washed 4× with PBS containing 0.1% Tween20 (PBS-T) and detected by incubating 2 h30 min with Alexa conjugated secondary antibodies (Abcam). Secondary antibody was washed 5× with PBS-T. Nuclei were stained for 5 min with 300 nM DAPI (4′,6-diamidino-2-phenylindole) in PBS at RT. Slides were rinsed in PBS and mounted with Mowiol mounting media and microscope cover glass No. 1.5H (Marienfeld). Co-staining of F4/80 and MANF was performed without the permeabilization with Citrate buffer and blocked with Horse Serum (HS) 10% in PBS-T. Staining of necrotic myofibres using secondary antibody anti-mouse IgG coupled to Alexa-647 was performed without primary antibody incubation overnight. As myofibers become permeable during necrosis, the passive uptake of IgG proteins allows for staining of necrotic fibers using anti-IgG antibodies

Imaging: Digital images were acquired at the Bioimaging and Comparative Pathology facilities of Instituto de Medicina Molecular using: (1) a digital slide scanner NanoZoomer SQ (HAMAMATSU), with an objective of 20× magnification, for H&E stained sections; (2) a motorized inverted widefield fluorescence microscope (Zeiss) equipped with CCD camera (Photometrics CoolSNAP HQ CCD), with a 20× objective for IHC stained sections (eMHC, IgG). The total stained sections after imaging were reconstructed through image overlay (10%); (3) a Zeiss LSM 710 confocal laser scanning microscope (F4/80/MANF IHC).

Image quantification: To assess the effectiveness of the muscle regeneration the individual new myofibres (eMHCpos) were manually outlined in the total muscle section and their cross-sectional area (CSA) was determined with the public domain image analysis software ImageJ. The number of necrotic fibres in total muscle section was quantified using the same software.

### Flow Cytometry (FC) Analysis

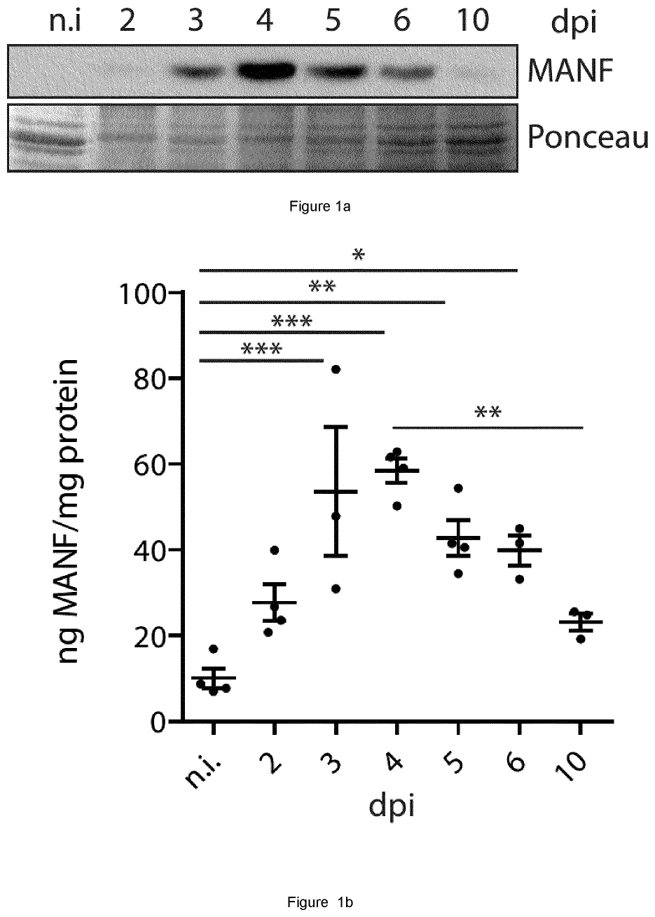

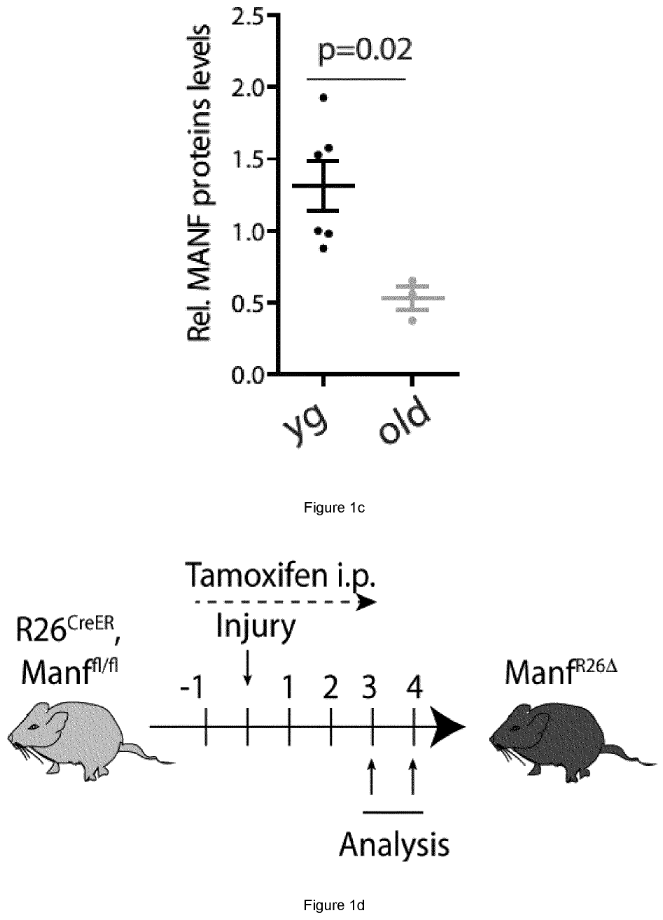

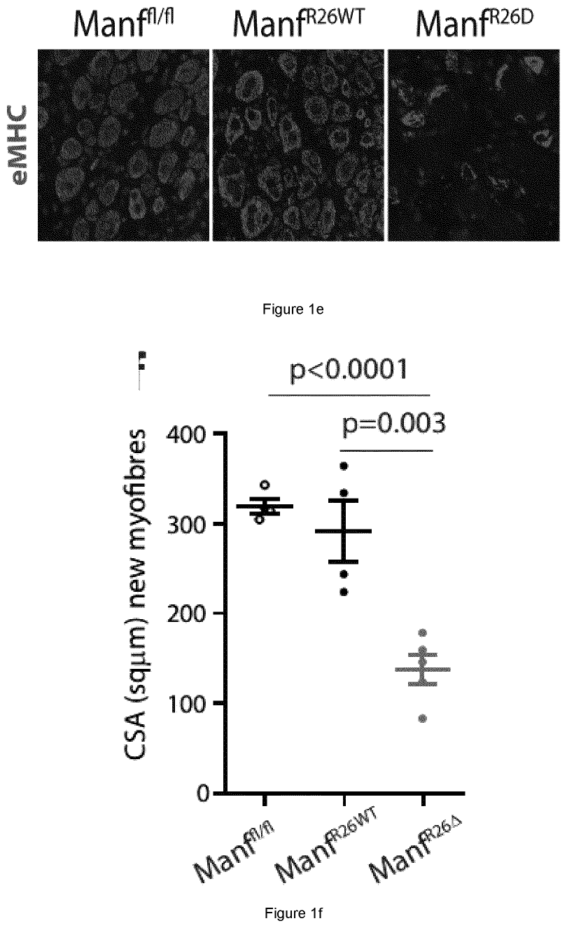

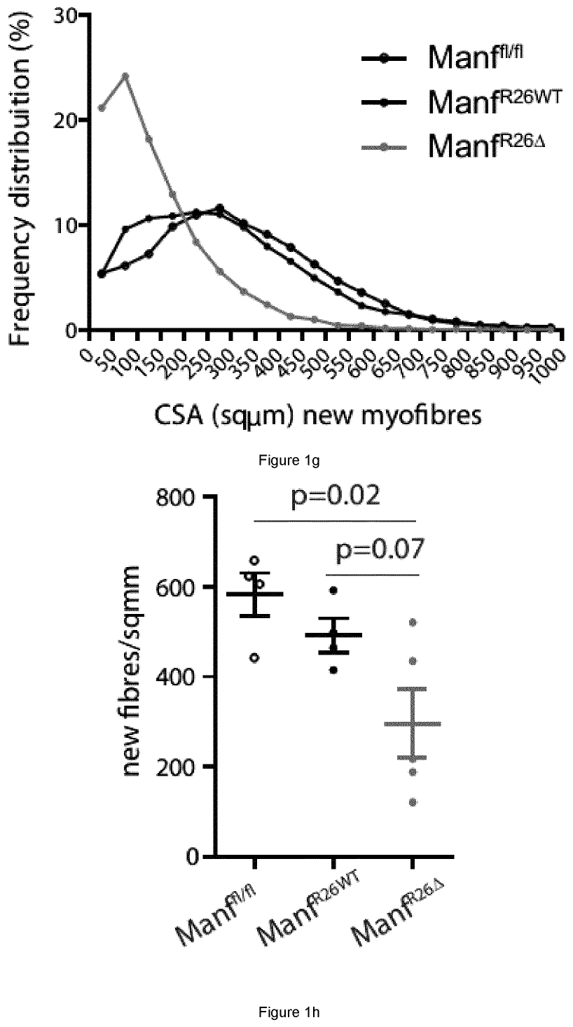

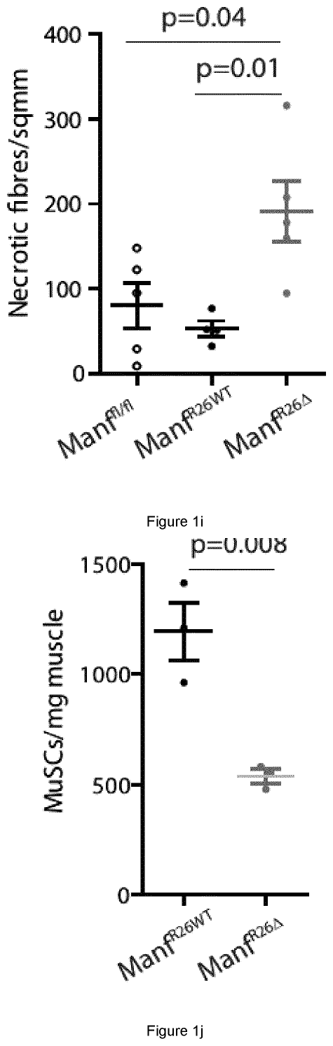

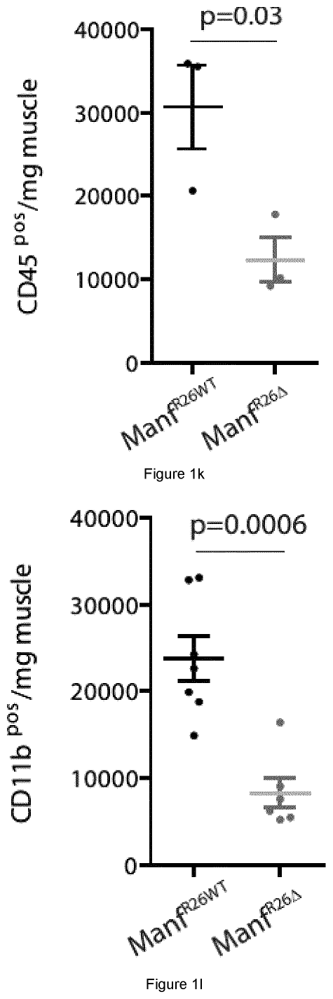

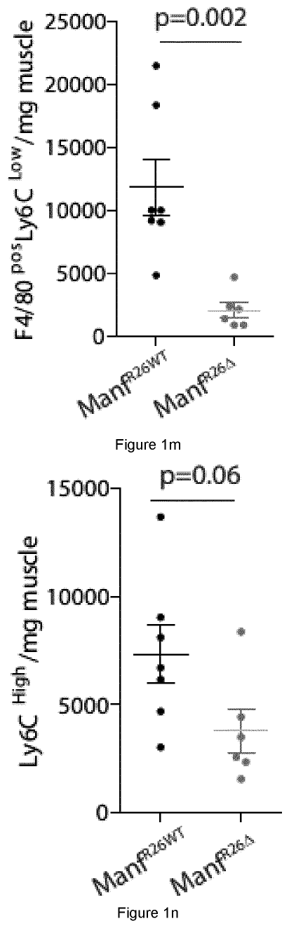

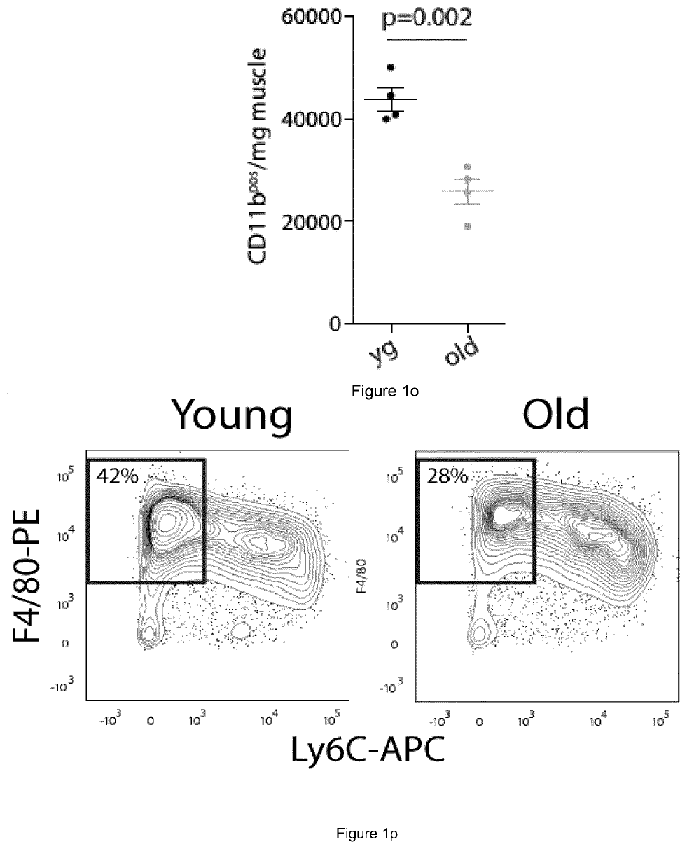

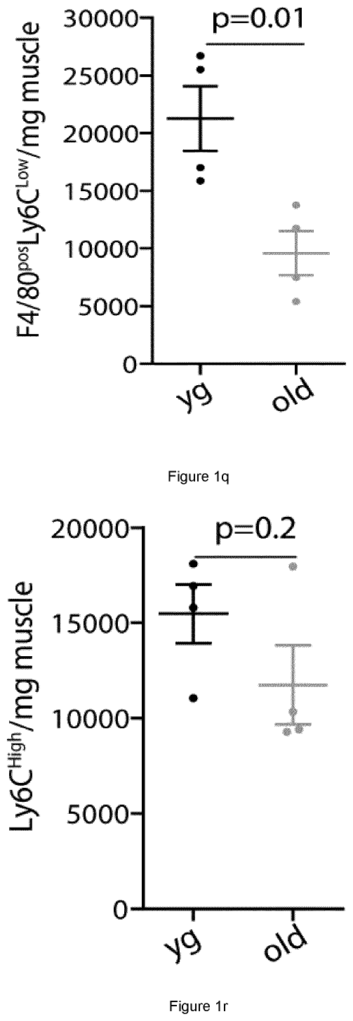

Muscle cell population analysis: To obtain single cell suspensions, muscles were mechanically disaggregated and dissociated in DMEM 1% P/S media containing collagenase B (Roche) 0.2% and Calcium dichloride (CaCl2) 0.5 mM at 37° C. for 1 h and then filtered through 70 μm cell strainers (Falcon). Cells were incubated in 1× Red Blood Cell (RBC) lysis buffer (Santa Cruz Biotechnology) for 10 min on ice, resuspended in 1 ml of DMEM 10% FBS 1% P/S media and counted. For flow cytometry analysis (FC analysis), single cell suspension samples were resuspended in PBS containing 5% HS with fluorophore-conjugated antibodies at a density of 1×106 cells/100 μl, incubating 30 min at 4° C., protected from light. Cells were re-suspended in PBS containing 5% HS for FC analysis. CD45, CD31 markers were used to exclude the Lin (−) negative population from single live cell population and the population of MuSCs and FAPS were identified as α7-integrinposand Sca-1pos, respectively. Live cells were identified using LIVE/DEAD™FixableNear-IR Dead Cell Stain (Invitrogen). Gating strategy used in FC analysis of CD45posimmune cell population, endothelial cells, FAPS and MuSCs is shown in Extended data FIG. 1f. Gating strategy used in FC analysis of myeloid cells (CD11bpos), pro-repair macrophages (F4/80posLy6C^{Low}), pro-inflammatory macrophages (Ly6C^{High}), and neutrophils (Ly6Gpos) is presented in Extended data FIG. 1i.

Proliferation analysis: Cell proliferation was determined by EdU detection using the EdU-Click 488 kit. Following staining of cell-surface antigens with antibodies as described above and cell fixation using 1× Intracellular Fixation Buffer (eBioscience™, 00-8222-49) in PBS containing 5% HS, cell suspensions were permeabilized in 100 ul of 1× Permeabilization Buffer (eBioscience™, 00-8333) in distilled water. Cells were incubated in a Click reaction for 30 min at RT protected from light, washed with 500 ul of Permeabilization buffer, resuspended in PBS containing 5% HS and immediately analyzed by FC. FMO control for EdU Detection was obtained using all the components for the click reaction except for the dye 6-FAM-Azyde. 15 Apoptosis analysis: Cell apoptosis was monitored by staining the cells with Apopxin Green solution from the Apoptosis/Necrosis Assay Kit (ab176749). Following the staining of cell-surface antigens with antibodies as described above, cells were resuspended in Assay Buffer with Apopxin Green Indicator at 1:100 and incubated for 45 min at RT. Apopxin Green Solution was not added to cells for FMO control.

Blood cell population analysis: Blood was collected by heart puncture and incubated twice in 1×RBC lysis buffer for 15 min at RT, with periodic inversions. Blood cells were incubated in viability dye Zombie Aqua (1:1000; Biolegend) diluted in PBS for 15 min on ice, blocked using anti-mouse CD16/32 FcγR (Biolegend) diluted at 1:250 in 1× Brilliant Stain Buffer Plus (BD Biosciences) in PBS-2% FBS (BV Buffer) for 15 min, and fluorophore-conjugated antibodies were added at a density of 5×105 cells/50 μL, incubated for 30 min at 4° C., and protected from light. Blood cells were resuspended in PBS containing 2% FBS for FC analysis. For information on the antibodies used, see Supplementary Table 3. Live blood cells were identified using the viability dye Zombie Aqua.

For all analyses, characterization of cell populations was performed at the Flow cytometry facility of Instituto de Medicina Molecular, using a cell analyzer LSRFortessa X-20 (BD Bioscience) with FACSDiva 8.0 software. Flow cytometry data were analyzed using FlowJo (BD Biosciences) analysis software.

### Fluorescence Activated Cell Sorting (FACS) of Macrophage Populations

Single cell suspensions, obtained as described above, were used to isolate macrophage populations through staining with fluorochrome-conjugated antibodies. CD45^{pos}F4/80^{pos} macrophages were selected from the viable cells present in the single cell suspension.

F4/80^{pos}Ly6C^{low} and LyC6Highmacrophages were isolated using the gating strategy presented in Extended data FIG. 1i. The isolation of pure populations of cells was performed at the Flow cytometry facility of Instituto de Medicina Molecular using a FACSAria Ilu (BD Bioscience) or a FACSAria III (BD Bioscience) using the software FACSDiva 6.1.3. Cells were collected in PBS containing 5% HS and used either for protein extraction, RNA extraction, TEM analysis or ex-vivo assays.

### Ex-Vivo Macrophage Analysis

Whole muscle single cell suspensions: Single cell suspensions of 2pi injured muscles were obtained as described above. For each animal, 500 000 cells were collected at 0 h or cultured and collected after 16 h. Cells were incubated in suspension at 37° C. in SF medium (Corning® SF Medium, with L-glutamine and 1 g/L BSA) supplemented with 10% FBS and 1% Pen/Strep. In conditions of MANF supplementation, rMANF (P-101-100, Icosagen) was used at a concentration of 10 μg/ml. Cells collected at 0 h and 16 h were stained for FC analysis of muscle immune populations as described above.

Sorted Ly6C^{High} macrophages: Ly6C^{High}macrophages were isolated by FACS from 2 dpi injured muscles, as described above, and 50.000 cells were collected for analysis at 0 h or cultured and collected for analysis after 16 h. Cells were incubated at 37° C. in DMEM medium supplemented with 10% FBS and 1% Pen/Strep. Cells collected at 0 h and 16 h were stained for flow cytometry analysis of muscle immune populations or used for RNA analysis. Ly6C-APC antibody was used during cell sorting and Ly6C-FITC antibody was used for detection of Ly6C at 0 h and 16 h in flow cytometry analysis.

### Transmission Electron Microscopy Analysis of Macrophages

Sample processing and imaging: Pro-repair macrophages were isolated by FACS as described above, plated on 12 mm coverslips inserted in a 24-well plate with DMEM media containing 1% PS and 10% FBS, and allowed to adhere for 2 h. Sample processing and imaging was performed at the Electron Microscopy facility at Instituto Gulbenkian de Ciênca (IGC, Lisbon, Portugal). Cells were fixed with 2% formaldehyde (FA)-2.5% Glutaraldehyde in 0.1M phosphate buffer (PB) for 45 min on ice and then fixed O/N in 1% FA in PB at 4° C. The following day, cells were washed 2× in PB, post-fixed with 1% osmium in PB for 1 h on ice, washed 2× in PB, 2× in water, stained with 1% tannic acid 20 min on ice, washed 2× in water, stained with 0.5% in Uranyl acetate 1 h at RT and serial dehydrated in increasing concentrations of ethanol. Coverslips with cells were mounted on top of EPON capsules and baked at 60° C. O/N. Sections of 70 nm were obtained using a UC7 Ultramicrotome (Leica) and stained with uranyl acetate and lead citrate for 5 minutes each.

Images of single macrophages were acquired on a Tecnai G2 Spirit BioTWIN Transmission Electron Microscope (TEM) from FEI operating at 120 keV and equipped with an Olympus-SIS Veleta CCD Camera. Image quantifications: Quantification of individual pro-repair macrophages from ManfCx3cr1Δ (n=42) and ManfCx3cr1WT (n=54) mice, analyzed by TEM, was performed using the ImageJ software, after scale normalization. Intracellular vesicles were manually surrounded, and the number and area were determined. In addition, the area of each cell was also calculated.

In Vitro Experiments with Bone Marrow-Derived Macrophages (BMDMs)

Preparation of BMDMs: BMDMs were differentiated in vitro from bone marrow (BM) progenitors. To isolate BM, both hindlimbs of each mouse were collected and the femur and tibia were individualized and kept on ice in sterile PBS with 1% P/S. Bone marrow was flushed out onto a plate using a 1 ml syringe with a 26G needle filled with sterile media DMEM 10% FBS 1% P/S. Red blood cell clumps were mechanically disrupted using the syringe, and the cell suspension was collected and filtered through a 70 μm filter, centrifuged at 500 g for 5 minutes, resuspended in 1 ml of 1×RBC lysis buffer and incubated at RT for 5 minutes and washed with 10 ml of PBS. Cells were diluted in complete media to achieve 106 cells/ml density and plated in 10 cm plates. Plated cells were supplemented with CSF-1 at 50 ng/ml (Biolegend) and cultured at 37° C. On the third day, media was changed and re-supplemented with CFS-1. On the sixth day, cells were detached and frozen in FBS 10% DMSO.

Preparation of Apop-necro: For Apop-Necro debris preparation, C2C12 myoblast cell line was used (ATCC, CRL1772). C2C12 cultures at 80% confluence were starved in PBS overnight to induce cells apoptosis. The following day, PBS and apoptotic cells in suspension were collected and counted. C2C12 suspension was centrifuged for 5 min at 3000G. Pellet was then frozen and stored at −80° C. until BMDM stimulation.

Lysosomal-activity assay in BMDMs: BMDMs were thawed and 200.000 cells were plated in 12 well plates in medium supplemented with 10% FBS and 1% Pen/Strep and incubated overnight at 37° C., in the absence of CSF-1. Lysosomal intracellular activity was evaluated using the Lysosomal Intracellular Activity Assay Kit (Abcam, ab234622). For the assay, cells were incubated with a self-quenched substrate in DMEM supplemented with 0.5% FBS and 1% Pen/Strep for 3 h, according to manufacturers' instructions. Basal activity was accessed in the absence of any co-stimulation. Phagocytosis-associated lysosomal activity was assessed during co-stimulation with apop-necro debris at a proportion of 2:1. Frozen apoptotic cells were thawed immediately before stimulation to induce additional necrosis. Bafilomycin A was used as a control to inhibit lyosomal activity in both conditions. In conditions of MANF supplementation, rMANF (P-101-100, Icosagen) was used at a concentration of 10 μg/ml during the assay. Lysosomal hydrolysis of the self-quenched substrate was measured by flow cytometry through the quantification of fluorescence intensity in the FITC channel.

Bead phagocytosis assays in BMDMs: BMDMs were thawed and 100.000 cells were plated in 24 well plates in medium supplemented with 10% FBS and 1% Pen/Strep and incubated overnight at 37° C., in the absence of CSF-1. Evaluation of phagocytic uptake capacity was performed using Fluoresbrite®641 Carboxylate Microspheres (Polysciences, 17797-1). Opsonization of the particles was performed by incubation in 50% FBS in PBS for 30 min at 37° C. Opsonized particles were added to the BMDMS at a concentration of 5×108/ml and incubated for 3 h. Phagocytic uptake was measured by flow cytometry through the quantification of fluorescence intensity of the Fluoresbrite®641 within the BMDM single cell population.

### Protein Analysis

Preparation of muscle protein extracts: Whole muscle samples, or cells obtained from FACS, were homogenized in Lysis buffer (50 mM Tris pH 7.5, 150 mM NaCl, 0.5% NP-40, 5 mM EDTA, 1% Triton, in Mili-Q water) supplemented with protease inhibitors and phosphatase inhibitors (Sigma) for 45 min or 20 min at 4° C., respectively. The supernatant protein extracts were recovered by centrifugation and protein concentration in samples was determined using Bradford Reagent (VWR).

Western blot: Western blot analyses were performed on 12% SDS-PAGE. After electrophoretic separation, proteins were transferred onto nitrocellulose membranes using a Trans Blot Turbo Transfer system (BioRad). Membranes were blocked with Tris-buffered saline-0.1% Tween 20 (TBS-T) containing 5% milk for 1 h and incubated overnight at 4° C. with primary antibodies. Membranes were then incubated 1 h with a peroxidase-conjugated secondary antibody (1:10000; Abcam), and developed using Pierce ECL Western blotting substrate (ThermoScientific) or Clarity™Western ECL Substrate (BioRad). Ponceau S solution (Sigma) was applied to the nitrocellulose membranes before the blocking step to assess the total protein present.

Enzyme-Linked Immunosorbent Assay (ELISA): MANF concentrations in muscle tissue samples were quantified using an in-lab mouse MANF (mMANF) ELISA62. mMANF ELISA recognized both mouse and human MANF but did not recognize MANF homolog CDNF or give signal from tissue lysates from Manf^{-/-} mice, indicating that it was specific for MANF. Dynamic range of mMANF ELISA was 62.5-1000 μg/ml and its sensitivity 29 μg/ml. For mMANF ELISA measurement, muscle lysates from young mice were diluted at 1:500 in blocking buffer (1% casein in PBS-0.05% Tween-20), and lysates from young and old mice (2 dpi, 3 dpi) at 1:200, respectively. Recombinant human MANF (P-101-100; Icosagen) was used as a standard. All samples were measured in duplicate.

### RNA Analysis

Preparation of RNA samples: Total RNA from frozen muscle samples was extracted using TRIzol (Invitrogen), according to the supplier's instructions. Total RNA from sorted cells was extracted using RNeasy Micro kit (Qiagen), according to the supplier's instructions.

Reverse Transcription and real-time qPCR (RT-qPCR): Complementary DNA (cDNA) was synthesized using iScript cDNA synthesis kit (BioRad). Real-time PCR was performed on ViiA 7 Real-Time PCR System (Thermofisher Scientific), using Powerup SYBR Green MASTER MIX (Applied Biosystems). Expression of specific genes in each sample was normalized to beta-actin and results are shown as gene expression levels relative to levels in control samples which are arbitrarily set to one.

RNA sequencing and bioinformatics analysis: Library preparation, RNA sequencing, read mapping and FPKM quantification was performed as a service at Novogene (Cambridge, UK). RNA samples prepared as described above, from sorted macrophages (Manf^{Cx3cr1Δ}vs Manf^{Cx3cr1WT}) or pro-repair macrophages (Yg vs. old) were shipped to Novogene. Novogene team was responsible for Illumina library preparation (poly A enrichment) and sequencing using a NovaSeq instrument to generate 150 bp pair-end reads with an output of 6G per sample. Gene Ontology and KEGG analysis was carried out using The Database for Annotation, Visualization and Integrated Discovery (DAVID, https://david.ncifcrf.gov/).

### Statistical Analysis

All data are presented as average and standard error of the mean (s.e.m.). Statistical analysis was carried out using GraphPad Prism 5. For comparisons between two groups, a two-tailed Student's test was used to determine statistical significance, assuming normal distribution and equal variance. For multiple comparisons, one-way ANOVA with Bonferroni's multiple comparison post-test were used to determine statistical significance

### Results

### MANF is Essential for Muscle Regeneration

To explore the involvement of MANF in muscle regeneration, we evaluated MANF expression following muscle injury. We observed a sharp increase in MANF levels that peaked at 3-4 days post injury (dpi) and progressively declined thereafter (FIG. 1a-b). Importantly, this injury-dependent induction of MANF was blunted in aged animals (FIG. 1c), revealing an inability of aged muscles to significantly induce MANF between 2 and 3 dpi.

To understand the consequences of defective MANF signalling for muscle regeneration, we generated a mouse model allowing the inducible and ubiquitous ablation of MANF in adult animals. In the Rosa26-Cre^{ER}, Manf^{fl/fl} mice, tamoxifen treatment immediately before muscle injury and during muscle regeneration (FIG. 1d) resulted in a complete loss of MANF protein in the regenerating tissue. Analysis of tamoxifen-treated Rosa26-Cre^{ER}, Manf^{fl/fl} mice (herein referred to as Manf^{R26Δ}), revealed impairments in muscle regeneration compared to oil treated mice (Manf^{R26WT}) and tamoxifen-treated Manf^{fl/fl} mice. This is evidenced by a reduction in the density and the cross-sectional area (CSA) of new myofibres formed at 4 dpi (FIG. 1e-h) and a persistence of necrotic myofibres within the regenerating muscle (FIG. 1i), resembling the phenotype found in aged animals^{22,23}.

Manf^{R26Δ} mice also displayed a significant reduction in the number of MuSCs (p<0.01, FIG. 1j) and CD45^{pos} immune cells (p<0.05, FIG. 1k) in the regenerating muscle at 3 dpi, with no changes in the populations of fibroadipogenic progenitors (FAPs) or endothelial cells. Considering the fundamental role of myeloid cells in the clearance of necrotic debris, a process impaired in ManfR26□mice, we focused our analysis on the repair-associated myeloid response. Manf^{R26Δ} mice had a 65% reduction in the number of myeloid cells (CD11b^{pos}) at 3 dpi, corresponding to an 82% reduction in the pro-repair (F4/80^{pos}Ly6C^{Low}) macrophage population and a less pronounced reduction in the pro-inflammatory (Ly6C^{High}) population (p=0.06, FIG. 1l-n). The neutrophil population was unchanged. Thus, MANF loss is associated with defects in the repair-associated myeloid response characterized by a reduced presence of myeloid cells in the injured muscle and an imbalance in macrophage states, whereby pro-inflammatory macrophages tend to accumulate at the expense of pro-repair macrophages.

### Ageing Impairs the Repair-Associated Myeloid Response

Skeletal muscle from aged animals exhibits regenerative defects similar to those found in Manf^{R26Δ} mice^{22,23} and a blunted induction of MANF following injury (FIG. 1c). Thus, we asked whether this reduction in MANF levels in aged muscles was associated with similar defects in the repair-associated myeloid response. We found that 23-25 mo old animals had a 41% reduction in the number of myeloid cells within the regenerating skeletal muscle at 3 dpi (FIG. 10 and Extended data 1k), with a 55% reduction in the number of pro-repair macrophages and no significant changes in the number of pro-inflammatory macrophages (FIG. 1p-r). Importantly, these defects manifested as a reduced capacity to increase the total number of myeloid cells and pro-repair macrophages between 2 and 3 dpi. Thus, aged muscles display defects in myeloid cell accumulation and an imbalance of macrophage subpopulations that can be recapitulated in conditions of MANF loss. This parallel in myeloid defects included only the macrophage populations and not the neutrophil population, which was reduced in aged animals but not in Manf^{R26Δ} mice.

### MANF is Specifically Expressed in Pro-Repair Macrophages During Muscle Regeneration

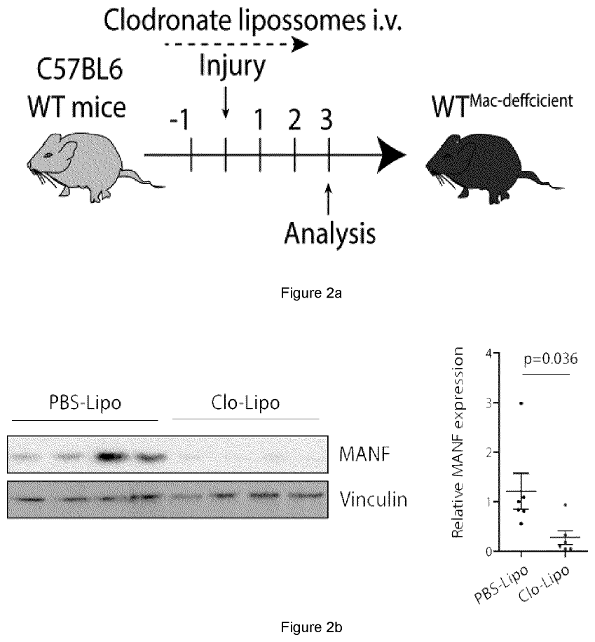

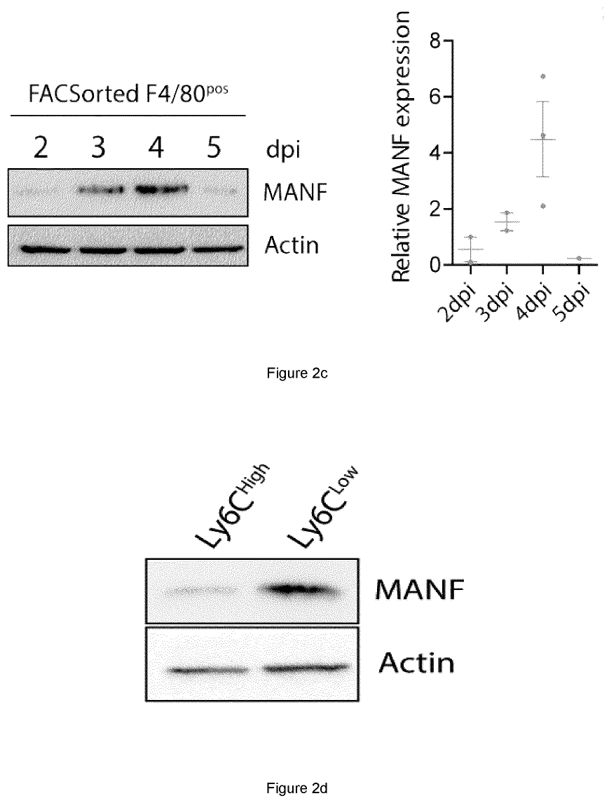

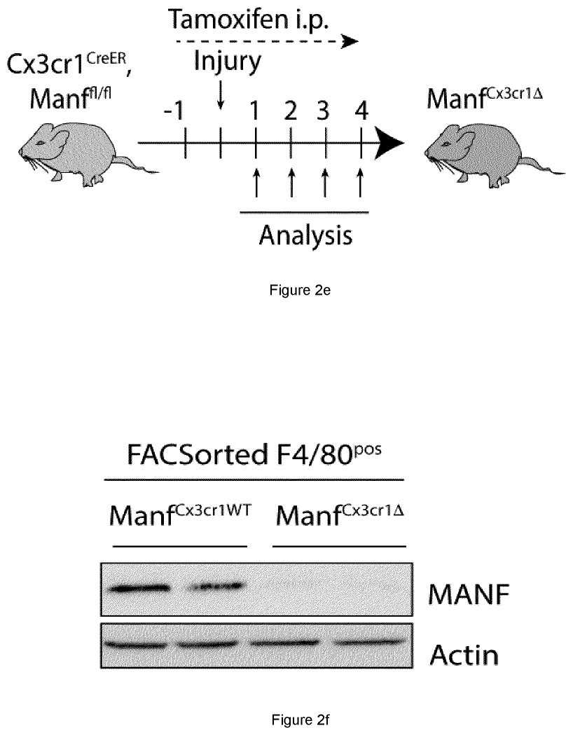

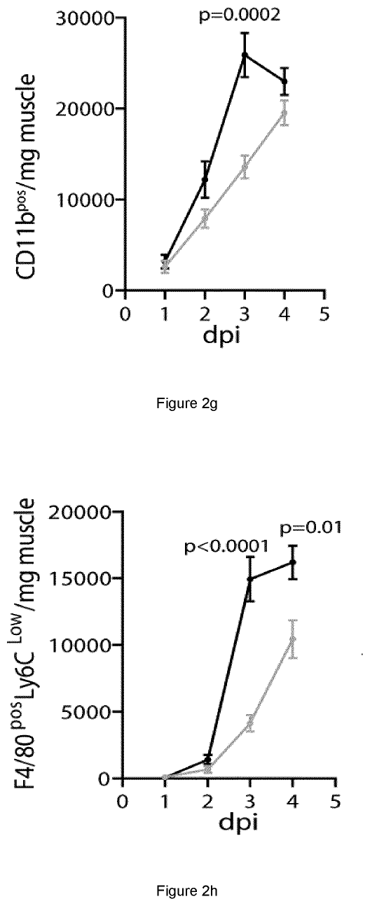

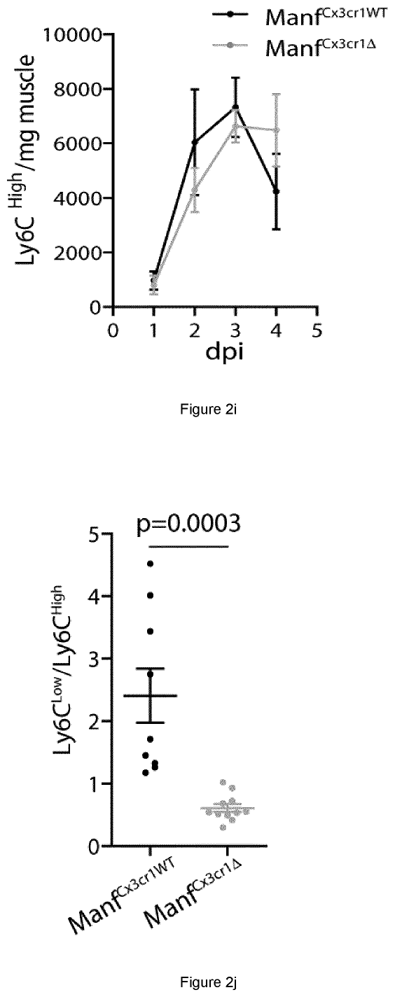

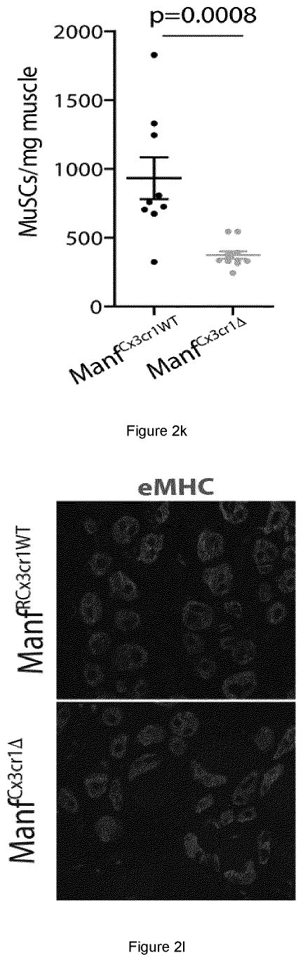

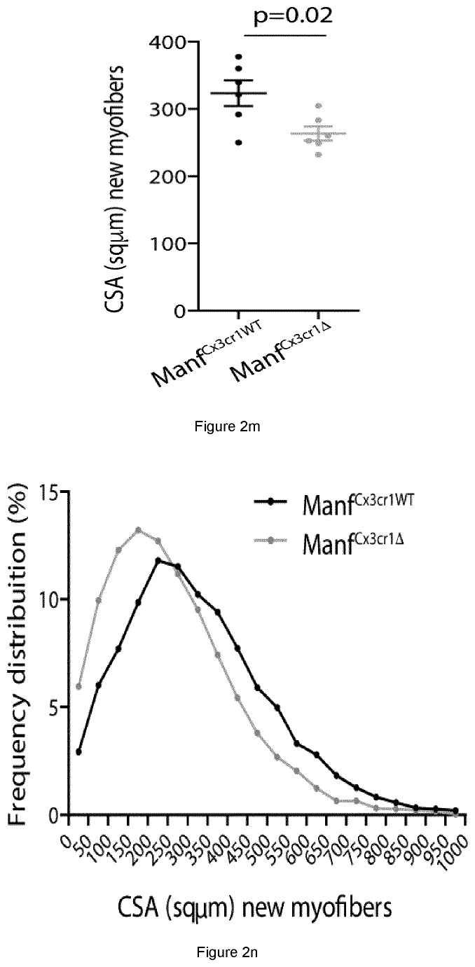

Since MANF is induced in macrophages after injury in other systems^{12,15} and macrophage numbers increase in the skeletal muscle following injury, we hypothesized that macrophages could be a source of MANF in the regenerating skeletal muscle. Indeed, F4/80^{pos}cells immunostained in muscle cryosections co-localized with sites of highest MANF expression. To further test this hypothesis, we treated mice with clodronate liposomes during muscle injury, generating a condition where macrophage numbers are reduced, but neutrophils are not affected (FIG. 2a), and observed a significant reduction in the levels of MANF protein present in the skeletal muscle at 3 dpi (p<0.05, FIG. 2b). To distinguish whether the increase in MANF levels is due to the influx of macrophages following muscle injury or the induction of MANF within the macrophage population, we isolated F4/80^{pos}cells by fluorescence activated cell sorting (FACS) and analyzed MANF protein expression. Interestingly, MANF protein levels were also changed in the F4/80^{pos}population of macrophages (FIG. 2c), mimicking the expression dynamic observed in whole muscles (FIG. 1a-b) and following the phenotypic transition of pro-inflammatory macrophages into pro-repair macrophages during muscle repair. Consistently, analysis of isolated macrophage subpopulations revealed that MANF is specifically induced in the F4/80^{pos}Ly6C^{low} subpopulation of pro-repair macrophages (FIG. 2d).

To confirm that this population is the main source of MANF during muscle regeneration we generated a mouse model to selectively deplete MANF in the emerging population of pro-repair macrophages that specifically express Cx3cr1²⁷. Indeed, tamoxifen-treated Cx3cr1-CreER, Manf^{fl/fl} mice (herein referred to as ManfCx3cr1Δ), displayed a complete ablation of MANF protein within the F4/80pospopulation of macrophages (FIG. 2e-f) and an 80% reduction in MANF levels in whole muscles when compared to oil treated mice (Manf^{Cx3cr1WT}). These data support the idea that MANF induction following muscle injury is not only due to an influx of macrophages to the site of injury, but mostly, to a specific increase in MANF protein levels associated with the emergence of pro-repair macrophages within the injured muscle.

### MANF Ablation in Cx3cr1posmacrophages Impairs Muscle Regeneration

To understand the consequences of MANF loss in Cx3cr1^{pos} macrophages, we analyzed Manf^{Cx3cr1Δ} animals on a time course following muscle injury, evaluating the repair-associated myeloid response and the efficiency of regeneration. Analysis of myeloid cell populations in Manf^{Cx3cr1Δ} mice revealed alterations in the dynamics of the transition between macrophage phenotypes and a reduced presence of myeloid cells within the skeletal muscle (FIG. 2g-j). These alterations were characterized by a significant reduction in the number of pro-repair macrophages (p<0.0001 at 3 dpi, FIG. 2h), and no alterations in the numbers of pro-inflammatory macrophages (FIG. 2i), causing a reduction in the ratio of pro-repair to pro-inflammatory macrophages at 3 dpi (FIG. 2j). Importantly, changes in myeloid populations were not due to the tamoxifen treatment, as they were still observed when Manf^{Cx3cr1Δ} mice were compared with tamoxifen-treated Manf^{fl/fl} mice as controls.

Evaluation of regenerating muscles revealed that MANF ablation in Cx3cr1pos macrophages was sufficient to cause defects in muscle repair, evidenced by a reduction in the number of MuSCs present at 3 dpi (FIG. 2k), reduced CSA of new myofibres and increased presence of necrotic fibres at 4 dpi, that persisted at 14 dpi (FIG. 2l-o), despite no defects being detected prior to injury. Thus, MANF derived from Cx3cr1^{pos} macrophages is essential for a regulated myeloid response, successful debris clearance following muscle injury, and effective muscle regeneration.

MANF is Essential for a Timely Phenotypic Transition of Macrophages into the Pro-Repair State

Interestingly, the defects in the repair-associated myeloid response observed in Manf^{Cx3cr1Δ} animals were not detected at 1 dpi and developed primarily between 2 dpi and 3 dpi (FIG. 2g-j), suggesting that they are associated with specific mechanisms operating during the process of transition of macrophage phenotypes. Indeed, tamoxifen treatment of Cx3cr1-Cre^{ER}, Manf^{fl/fl} mice only prior to the injury (and not during the injury) did not result in any defects in the repair-associated myeloid response, supporting the idea that the defects observed are due to MANF-ablation in Cx3cr1poscells that appear only during the injury and not Cx3cr1^{pos}resident macrophages or Cx3cr1posnon-classical monocytes. Consistently, we did not observe any alterations in myeloid cell composition in the skeletal muscle of Manf^{Cx3cr1Δ} animals in homeostasis, nor in the blood of Manf^{Cx3cr1Δ} animals in homeostasis or after injury. Moreover, proliferation (evaluated by EdU incorporation between 2 and 3 dpi), and apoptosis (evaluated by Apopxin Green indicator) was not different in the macrophage populations present in the skeletal muscle at 3 dpi. This analysis supports the idea that the myeloid defects observed in Manf^{Cx3cr1Δ} animals are likely associated with impairment in the process of phenotypic transition in the macrophage population.

To explore the potential role of MANF in macrophage's phenotypic transition ex vivo, we generated an additional mouse model where MANF is ablated in macrophages in a tamoxifen-independent manner (Manf^{LysMΔ} mice). Similarly to Manf^{Cx3cr1Δ} mice, Manf^{LysMΔ} mice had lower numbers of myeloid cells within the regenerating muscle and a delayed transition between macrophage phenotypes when compared to Manf^{fl/fl} mice.

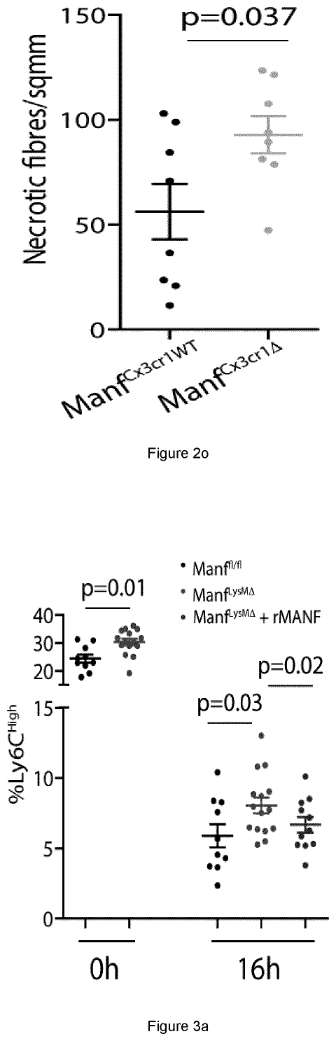

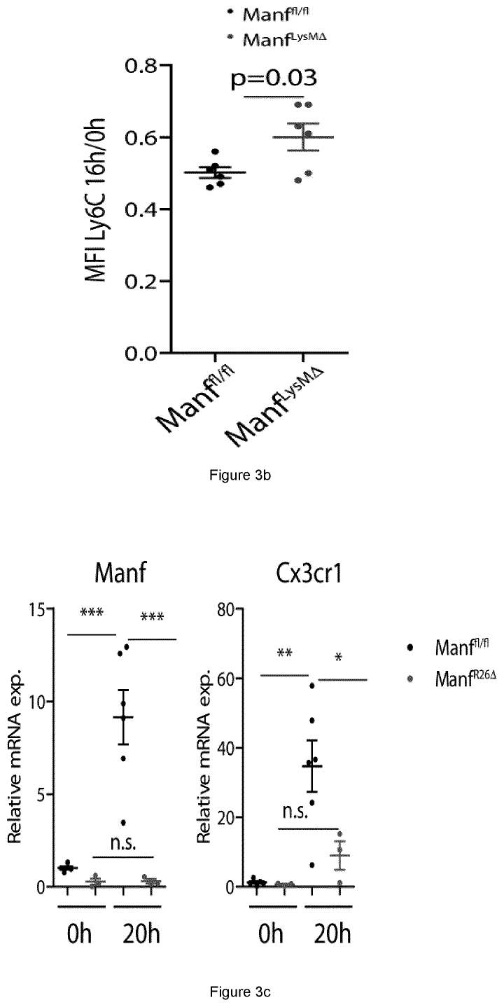

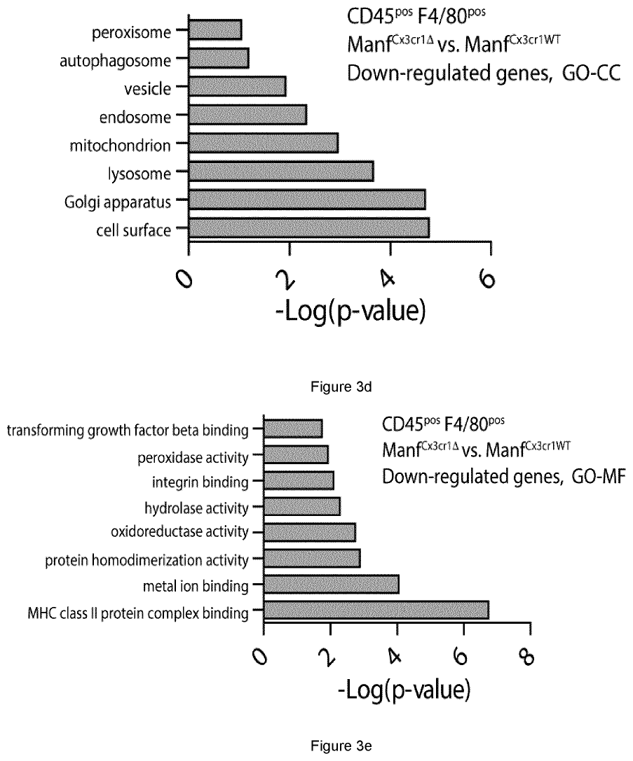

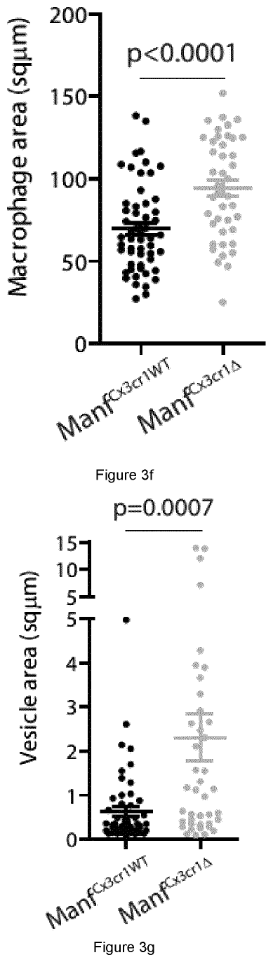

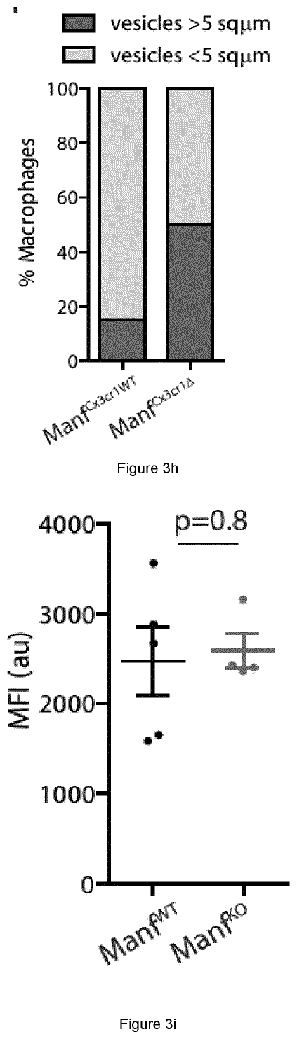

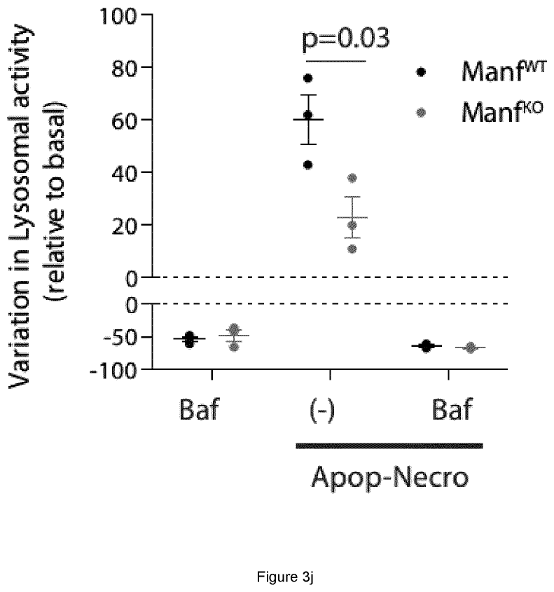

To evaluate a specific role of MANF in macrophages' phenotypic transition, we developed an ex vivo assay that allows us to follow the transition of pro-inflammatory macrophages into pro-repair macrophages, independently of the alterations in myeloid cell numbers. In this assay, single cell suspensions isolated from 2 dpi muscles were cultured for 16 h and the distribution of macrophage populations was quantified by flow cytometry at 0 h and 16 h. Cultured macrophages recapitulated the phenotypic transition observed in vivo, which can be quantified by the loss of Ly6C marker. Indeed, Manf^{LysMΔ} mice showed defects in the process of phenotypic transition ex-vivo, evidenced by a higher percentage of cells retaining the Ly6C^{High} status after 16 h in culture (FIG. 3a). Importantly, these defects could be ameliorated by supplementing recombinant MANF (rMANF) protein in the culture media (FIG. 3a), suggesting a feed-forward autocrine mechanism by which MANF derived from pro-repair macrophages sustains the formation of new pro-repair macrophages. Since the percentage of Ly6C^{High} macrophages is already higher at 0 h in Manf^{LysMΔ} mice it is possible that the differences we observed at 16 h were also a reflection of this initial delay. Thus, we repeated this experiment using Ly6C^{High} macrophages isolated by FACS. Indeed, Ly6C^{high} macrophages from Manf^{LysMΔ} mice showed significant impairments in performing the phenotypic transition when compared to Man^{fl/fl} mice, evaluated by the ability to downregulate Ly6C expression (FIG. 3b). This observation was further confirmed in Ly6C^{high} macrophages isolated by FACS from Manf^{R26Δ} mice: RNA analysis of macrophages followed for 20 h in culture after sorting showed that macrophages isolated from tamoxifen-treated Man^{fl/fl}mice up-regulate Manf and Cx3cr1, indicative of an efficient process of phenotypic transition that parallels what is observed in vivo. On the contrary, macrophages isolated from Manf^{R26Δ} mice had negligible levels of Manf expression and showed significant defects in the induction of Cx3cr1 (FIG. 3c). Finally, intrinsic defects in MANF-deficient macrophages were also confirmed in bone marrow-derived macrophage (BMDM) cultures generated from Manf^{R26WT} and Manf^{R26Δ} mice. Manf^{KO} macrophages had higher levels of pro-inflammatory genes and induced a stronger pro-inflammatory response after stimulation with Fibrinogen, a common signal present during muscle regeneration. Collectively, this analysis support the idea that that MANF is an essential autocrine regulator of macrophages' phenotypic transition during muscle regeneration and is important to limit excessive pro-inflammatory signalling.

MANF-Deficiency is Associated with Defects in Phagocytosis-Associated Lysosomal Activity

RNA sequencing (RNAseq) analysis of macrophages (CD45^{pos}F4/80^{pos}) freshly isolated from Manf^{Cx3cr1Δ} and Manf^{Cx3cr1wt} muscles at 3 dpi revealed changes in key cellular processes (FIG. 3d-e). Gene ontology analysis of the dataset of down-regulated genes revealed enrichment for genes associated with lysosomal and endosomal compartments (FIG. 3d), and with molecular functions related to hydrolase, peroxidase and oxidoreductase activity (FIG. 3e). Consistently, functional classification of all differentially expressed genes revealed alterations in the biological processes of MHC class II antigen presentation, a cellular process dependent on the efficient lysosomal digestion of internalized proteins by phagocytosis, and in the response to oxidative stress. Additionally, functional classes of biological functions associated with normal functions of pro-repair macrophages were also altered in Manf-deficient macrophages, suggesting functional alterations in the context of tissue repair.

To better characterize the alterations of Manf-deficient macrophages we zoomed in on the pro-repair population. The F4/80^{pos} Ly6C^{Low} subpopulation of pro-repair macrophages was isolated by FACS from Manf^{Cx3cr1Δ} and Manf^{Cx3cr1wt} muscles at 3 dpi and analyzed by transmission electron microscopy (TEM). Manf-deficient pro-repair macrophages exhibited marked structural differences (FIG. 3f-h), characterized by a significant increase in size (p<0.0001, FIG. 3f) and an accumulation of large vesicular structures, often filled with undigested cellular material (FIG. 3g-h). These structural alterations are consistent with the gene expression changes associated with alterations in the lysosomal compartment and hydrolytic activity and may reflect alterations in the phagocytic pathway, previously associated with the phenotypic transition of macrophages into a pro-repair state¹³.

To specifically test whether Manf-deficient macrophages showed defects in phagocytic activity we performed in vitro experiments using bone marrow-derived macrophages (BMDMs) generated from Manf^{26Δ} mice and measured their capacity to uptake opsonized beads and to increase their lysosomal hydrolytic activity upon stimulation with cellular debris. We could not detect any defects in cargo uptake capacity in MANF-deficient macrophages (FIG. 3i). However, while macrophages that express MANF increased their basal lysosomal hydrolytic activity by about 60% upon stimulation with cellular debris, MANF-deficient macrophages only increased their lysosomal activity by about 30%, evidencing significant (p=0.03) defects in phagocytosis-associated lysosomal hydrolysis (FIG. 3j).

### MANF Restores the Repair Associated Myeloid Response and Improves Muscle Regeneration in Ageing

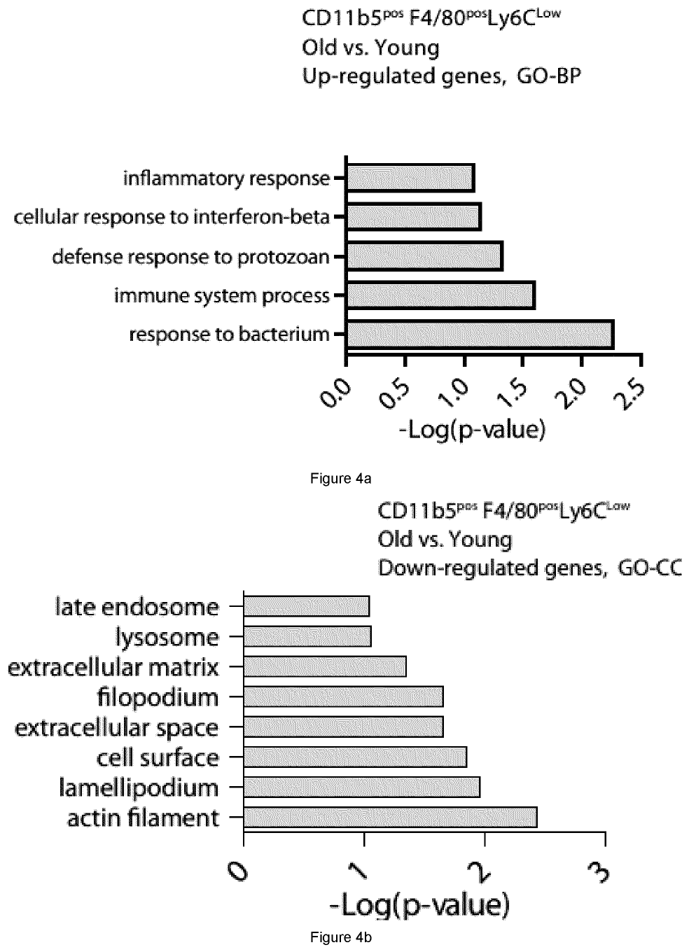

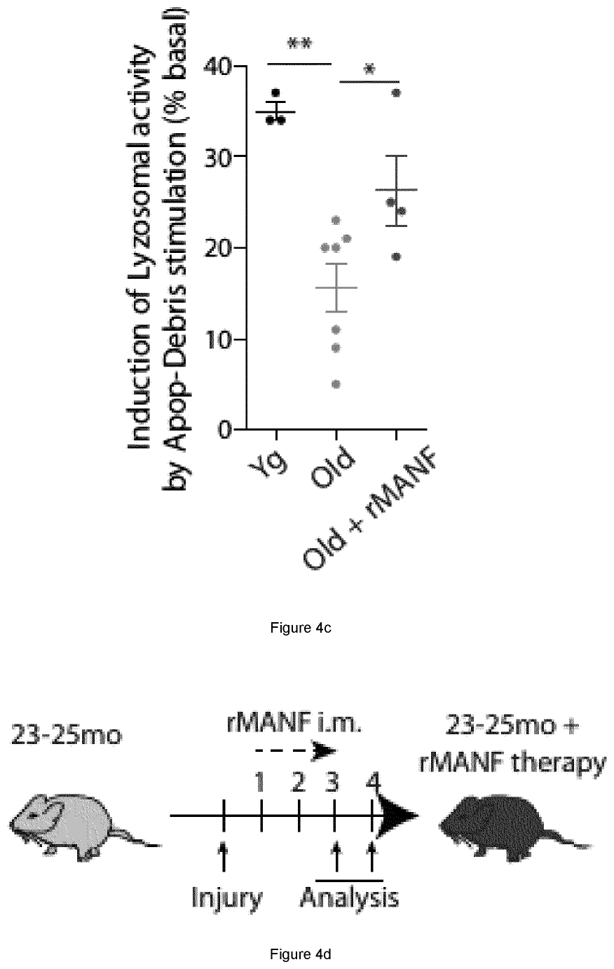

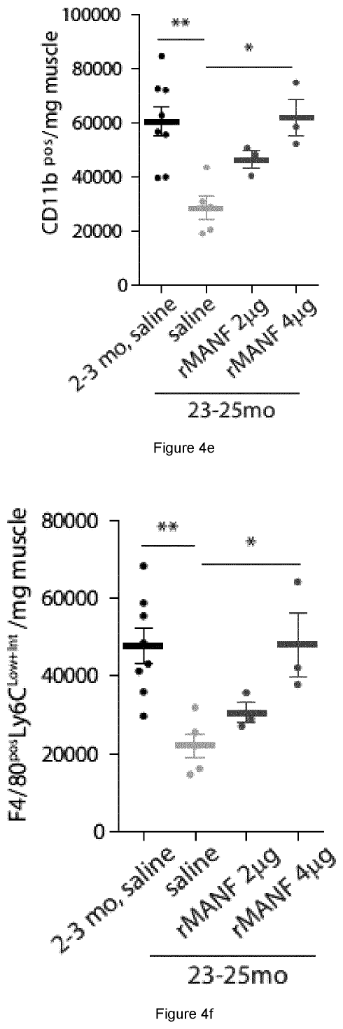

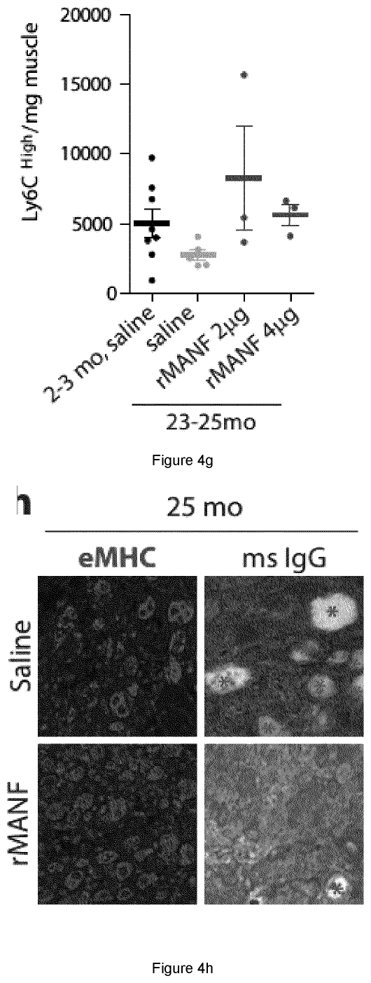

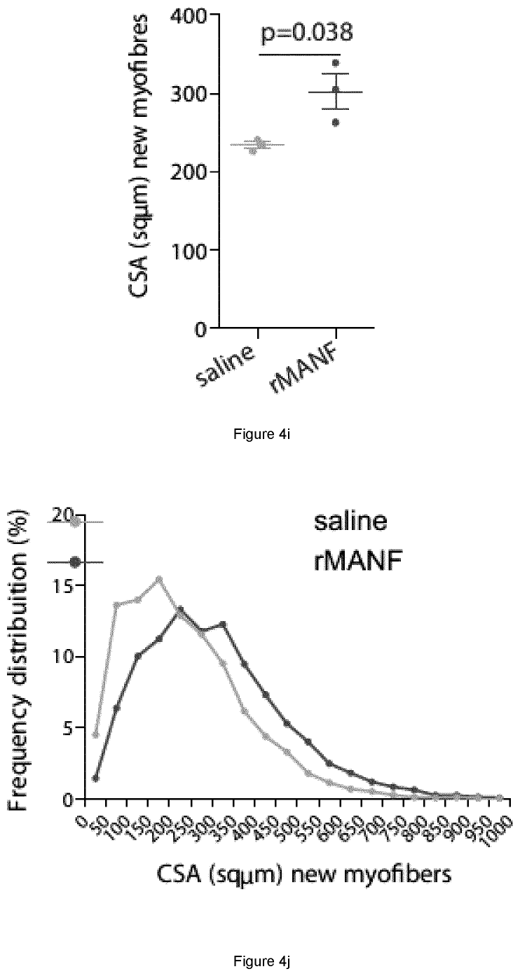

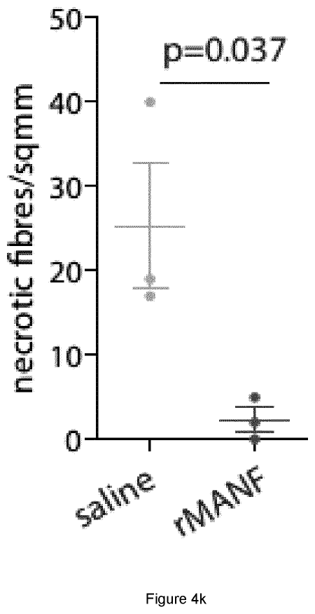

To understand if similar defects were present in old animals, we performed RNAseq analysis of pro-repair macrophages (CD11b^{pos}F4/80^{pos}Ly6C^{Low}) isolated from aged muscles at 3 dpi. The dataset of up-regulated genes was enriched for gene ontologies associated with inflammatory activation, suggesting a shift in the gene expression profile of the pro-repair population towards a pro-inflammatory phenotype (FIG. 4a). Similarly to what we observed in MANF-deficient macrophages, the dataset of down-regulated genes revealed enrichment in gene ontologies of cellular components associated with lysosomal and endosomal compartments, but also changes associated with filopodia and lamellipodia, not present in MANF-deficient macrophages (FIG. 4b). Genes down-regulated in aged macrophages also classified in biological processes with relevance within the context of tissue regeneration, such as wound healing, endothelial cell morphogenesis or regulation of cell-cell adhesion. Interestingly, in vitro assays using BMDMs derived from yg and aged mice, showed that aged macrophages also manifest defective phagocytosis-induced lysosomal activity, which could be improved by rMANF supplementation (FIG. 4c).

Since MANF induction following injury was impaired in aged animals (FIG. 1c); defects in the repair-associated myeloid response and muscle regeneration were similar in aged and MANF-deficient mice (FIG. 1d-r); and MANF supplementation was sufficient to restore pro-repair macrophages in models of MANF deficiency (FIG. 3a) and improve lysosomal activity in phagocytic aged macrophages (FIG. 4c), we sought to explore whether MANF therapy could allay the age-related defects in muscle regeneration.

Our strategy consisted in delivering rMANF through daily intramuscular (i.m.) injections (2 or 4 ug/injections) starting at 1 dpi and up to the day of analysis (FIG. 4d). We found that delivery of rMANF to injured muscles of aged mice was sufficient to normalize the repair-associated myeloid response, restoring the numbers of myeloid cells and pro-repair macrophages at 3 dpi (FIG. 4e-g). The effects were dose-dependent and MANF therapy at 4 ug/i.m. injection resulted in a complete rescue of the repair-associated myeloid response. Importantly, the same regimen of MANF therapy improved muscle regeneration in aged animals, resulting in an increase in the CSA of new myofibres and a reduction in the accumulation of uncleared necrotic fibres (FIG. 4h-k). These data show that restoring MANF levels during regeneration in aged muscles is sufficient to normalize the repair-associated myeloid response and suggest that immune modulatory interventions that rejuvenate macrophage function can be used to improve the regenerative capacity of aged muscles.

Age-related alterations in myeloid populations occur in the aged skeletal muscle, in homeostasis, with detrimental consequences for MuSC activity and tissue health²⁸⁻³¹. However, the effects of ageing on the myeloid response associated with muscle regeneration have just started to be explored^{31,32}. Here we uncovered a new regulator of the myeloid response to muscle injury, MANF, involved in the maintenance of a proper balance of macrophage populations throughout muscle regeneration. By identifying a critical down-regulation of MANF signalling in the aged regenerating skeletal muscle, we demonstrated how age-related changes in immune modulatory mechanisms contribute to the skeletal muscle's regenerative failure in ageing.

Previous studies have established that macrophages are essential for effective muscle repair, and murine models of macrophage ablation show defects in muscle regeneration characterized by accumulation of necrotic debris and defective myofiber formation^{13,33}, resembling the phenotype of MANF-deficient mice. The current knowledge regarding the role of macrophages on muscle regeneration is centred on the idea that macrophages act as signalling sources that control MuSC function, and intercellular communication defects are at the origin of muscle regenerative impairments in conditions of macrophage dysfunction¹². There are indeed several macrophage-derived signals that affect MusC activity, many of which altered during regeneration in ageing, including Klotho^{34,35}, GDF3³², CXCL10³⁶, osteopontin³⁷, among others³⁸. However, beyond their signalling activity, macrophages perform a central function of cellular debris clearance in the context of tissue injury. Indeed, phagocytic activity was recently shown to be essential for the phenotypic transition between macrophage populations^{13,39} and for effective muscle regeneration³⁹. Interestingly, the accumulation of necrotic debris in regenerating MANF-deficient mice is accompanied by defects in macrophage phenotypic transition. Thus, our data linking MANF function with phagocytosis-induced lysosomal activity may be the underlying mechanism through which MANF-loss leads to impairments in macrophage balance. Even though age-related immune signalling alterations have been linked with regenerative failure in aging³¹, it remains to be demonstrated whether re-establishing the immune cell function in aged animals is sufficient to improve regenerative capacity. Our results support the idea that direct modulation of macrophage function in ageing could be used to improve muscle regeneration. We also observed defects in the accumulation of myeloid cells following muscle injury in aged and MANF-deficient mice that warrant further investigation. The simultaneous rebalancing of macrophage populations and re-establishment of myeloid numbers following MANF delivery during regeneration raises the intriguing prospect that the two defects are linked. One possibility is that pro-repair macrophages are necessary for the secretion of a signal that sustains continuous recruitment of myeloid cells, whereas another is that accumulated debris produces and inhibitory signal that prevents further recruitment. Alternatively, considering its pleiotropic activities, it is possible that MANF may have an additional independent function, either local or systemic, not related to their direct activity in macrophages, which affects the efficiency of myeloid recruitment to the muscle.

MANF is an ubiquitous protein with multiple cellular targets reported^{16,18}. Although initially identified as a neurotrophic factor with cytoprotective activity on dopaminergic neurons^{40,41}, MANF has since been associated with additional functions in the retina^{15,42}, heart^{43,44}, liver^{20,45}, pancreas⁴⁶ and inner ear⁴⁷, some of which in the context of aging^{20,21,48}. Additionally, MANF's cytoprotective action now encompasses the engagement of tissue repair mechanisms through immune modulation^{15,48-50}. Indeed, our data provide indication that macrophages are the main source and target of MANF signalling during muscle repair. Nevertheless, the residual expression of MANF in macrophage depleted muscles, and the reduction in the magnitude of regenerative defects of our conditional Manf^{Cx3cr1Δ} relative to full Manf^{r26 Δ} model leave open the possibility of additional MANF sources in the regenerating muscle. It is also possible that beyond macrophages, other cellular targets of MANF in the muscle affect regenerative success, including the myofibre. This could be mediated by the regulation of the ER stress response, a cellular process associated with MANF signaling in multiple cell types^{51,52}, and required in the myofibre to support MuSC activity during regeneration⁵³. Alternatively, MANF could also act as a direct negative regulator of NFκB signaling in the myofibre⁵⁴, a mechanism previously implicated in the age-related loss of regenerative capacity⁵⁵.

Based on our assays, we propose that the effects we observe are mostly driven by an autocrine activity of MANF in macrophages. Addressing the question of whether blocking MANF response specifically in macrophages recapitulates the regenerative defects observed in our models is currently limited by our lack of knowledge regarding a MANF receptor in macrophages. Several possibilities can be explored to address this gap in knowledge: Neuroplastin has been proposed as a MANF receptor in other cell types, associated with the regulation of anti-inflammatory signaling⁵⁶. Interestingly, a recent report uncovered a role of Neuroplastin in macrophages, in the context of infection, connected with phagocytosis associated lysosomal function and chemotaxis⁵⁷. Additionally, co-localization of MANF and TLR receptors in innate immune cells has been observed in other model organisms⁵⁸ and may also be explored as a link between MANF function and phagocytic activity⁵⁹.

```
Sequences
SEQ ID NO: 1
    1 mrrmwatqgl avalalsvlp gsralrpgdc evcisylgrf yqdlkdrdvt fspatienel
  61 ikfcreargk enrlcyyiga tddaatkiin evskplahhi pvekiceklk kkdsqicelk
121 ydkqidlstv dlkklrvkel kkilddwget ckgcaeksdy irkinelmpk yapkaasart
181 dl
(NP_006001.5) MANF protein (Signal peptide (1-24) italics;
mature protein (25-182) normal)
SEQ ID NO: 2
    1 agtcggtcgg cggcggcagc ggaggaggag gaggaggagg aggatgagga ggatgaggag
  61 gatgtgggcc acgcaggggc tggcggtggc gctggctctg agcgtgctgc cgggcagccg
121 ggcgctgcgg ccgggcgact gcgaagtttg tatttcttat ctgggaagat tttaccagga
181 cctcaaagac agagatgtca cattctcacc agccactatt gaaaacgaac ttataaagtt
241 ctgccgggaa gcaagaggca aagagaatcg gttgtgctac tatatcgggg ccacagatga
301 tgcagccacc aaaatcatca atgaggtatc aaagcctctg gcccaccaca tccctgtgga
361 gaagatctgt gagaagctta agaagaagga cagccagata tgtgagctta agtatgacaa
421 gcagatcgac ctgagcacag tggacctgaa gaagctccga gttaaagagc tgaagaagat
481 tctggatgac tggggggaga catgcaaagg ctgtgcagaa aagtctgact acatccggaa
541 gataaatgaa ctgatgccta aatatgcccc caaggcagcc agtgcacgga ccgatttgta
601 gtctgctcaa tctctgttgc acctgagggg gaaaaaacag ttcaactgct tactcccaaa
661 acagcctttt tgtaatttat tttttaagtg ggctcctgac aatactgtat cagatgtgaa
721 gcctggagct ttcctgatga tgctggccct acagtacccc catgagggga ttcccttcct
781 tctgttgctg gtgtactcta ggacttcaaa gtgtgtctgg gattttttta ttaaagaaaa
841 aaaatttcta gctgtccttg cagaattata gtgaatacca aaatggggtt ttgccccagg
901 aggctccta
(NP_006010.6) MANF coding sequence (Signal peptide (53-124) italics;
mature peptide 125-598 normal)
```

### REFERENCES

1 Neves, J. et al. Cell stem cell 20, 161-175, doi: 10.1016/j.stem.2017.01.008 (2017).
2 Schultz, M. B. et al Development 143, 3-14, doi: 10.1242/dev.130633 (2016).
3 Conboy, I. M. et al. Nature 433, 760-764, doi: 10.1038/nature03260 (2005).
4 Conboy, I. M. et al Aging 7, 754-765, doi: 10.18632/aging. 100819 (2015).
5 Ruckh, J. M. et al Cell stem cell 10, 96-103, doi: 10.1016/j.stem.2011.11.019 (2012).
6 Neves, J. et al The FEBS journal 287, 43-52, doi: 10.1111/febs. 15061 (2020).
7 Yousefzadeh, M. J. et al. Nature 594, 100-105, doi: 10.1038/s41586-021-03547-7 (2021).
8 Wosczyna, M. N et al Developmental cell 46, 135-143, doi: 10.1016/j.devcel.2018.06.018 (2018).
9 Sousa-Victor, P. et al Nature reviews. Molecular cell biology 23, 204-226, doi: 10.1038/s41580-021-00421-2 (2022).
10 Munoz-Canoves, P. et al. The FEBS journal 287, 406-416, doi: 10.1111/febs. 15182 (2020).
11 Tidball, J. G . . . . Nature reviews. Immunology 17, 165-178, doi: 10.1038/nri.2016. 150 (2017).
12 Chazaud, B. Trends in immunology 41, 481-492, doi: 10.1016/j.it.2020.04.006 (2020).
13 Arnold, L. et al. The Journal of experimental medicine 204, 1057-1069, doi: 10.1084/jem.20070075 (2007).
14 Perdiguero, E. et al. The Journal of cell biology 195, 307-322, doi: 10.1083/jcb.201104053 (2011).
15 Neves, J. et al Science 353, aaf3646, doi: 10.1126/science.aaf3646 (2016).
16 Jntti, M. et al Cell and tissue research 382, 83-100, doi: 10.1007/s00441-020-03263-0 (2020).
17 Sousa-Victor, P., et al. Frontiers in physiology 9, 1629, doi: 10.3389/fphys.2018.01629 (2018).
18 Lindahl, M., et al. Neurobiology of disease 97, 90-102, doi: 10.1016/j.nbd.2016.07.009 (2017).
19 Tang, Q., et al TEM 33, 236-246, doi: 10.1016/j.tem.2022.01.001 (2022).
20 Sousa-Victor, P. et al Nature metabolism 1, 276-290, doi: 10.1038/s42255-018-0023-6 (2019).24
21 Neves, J. et al Experimental gerontology 134, 110893, doi: 10.1016/j.exger.2020.110893 (2020).
22 Sousa-Victor, P. et al Nature 506, 316-321, doi: 10.1038/nature13013 (2014).
23 Rahman, F. A et al International journal of molecular sciences 21, doi: 10.3390/ijms21134575 (2020).
24 Mircescu, M. M., et al. The Journal of infectious diseases 200, 647-656, doi: 10.1086/600380 (2009).
25 Kowalski, E. A. et al. JCI insight 7, doi: 10.1172/jci.insight. 156319 (2022).
26 Lehmann, M. L. et al Brain, behavior, and immunity 101, 346-358, doi: 10.1016/j.bbi.2022.01.011 (2022).
27 Varga, T. et al J Immunol 196, 4771-4782, doi: 10.4049/jimmunol. 1502490 (2016).
28 Wang, Y., et al Aging cell 17, e12828, doi: 10.1111/acel. 12828 (2018).
29 Wang, Y. et al. FASEB journal: official publication of the Federation of American Societies for Experimental Biology 33, 1415-1427, doi: 10.1096/fj.201800973R (2019).
30 Runyan, C. E. et al Aging cell 19, e13180, doi: 10.1111/acel. 13180 (2020).
31 Tobin, S. W. et al. Aging cell 20, e13312, doi: 10.1111/acel. 13312 (2021).
32 Patsalos, A et al. Aging cell 17, e12815, doi: 10.1111/acel. 12815 (2018).
33 Summan, M. et al. Regulatory, integrative and comparative physiology 290, R1488-1495, doi: 10.1152/ajpregu.00465.2005 (2006).
34 Sahu, A. et al. Nature communications 9, 4859, doi: 10.1038/s41467-018-07253-3 (2018).
35 Wehling-Henricks, M et al Human molecular genetics 27, 14-29, doi: 10.1093/hmg/ddx380 (2018).
36 Zhang, C. et al. Journal of cachexia, sarcopenia and muscle 11, 1291-1305, doi: 10.1002/jcsm.12584 (2020).
37 Paliwal, P., et al Aging 4, 553-566, doi: 10.18632/aging. 100477 (2012).25
38 Tidball, J. G., et al Experimental gerontology 145, 111200, doi: 10.1016/j.exger.2020.111200 (2021).
39 Al-Zaeed, N., et al Cell death & disease 12, 611, doi: 10.1038/s41419-021-03892-5 (2021).
40 Petrova, P. et al MN 20, 173-188, doi: 10.1385/jmn: 20:2:173 (2003).
41 Voutilainen, M. et et al The Journal of neuroscience: the official journal of the Society for Neuroscience 29, 9651-9659, doi: 10.1523/JNEUROSCI.0833-09.2009 (2009).
42 Lu, J. et al eNeuro 5, doi: 10.1523/ENEURO.0109-18.2018 (2018).
43 Glembotski, C. C. et al. The Journal of biological chemistry 287, 25893-25904, doi: 10.1074/jbc.M112.356345 (2012).
44 Zhang, Y. et al. Science translational medicine 14, eabo1981, doi: 10.1126/scitranslmed.abo1981 (2022).
45 He, M. et al World journal of gastroenterology 26, 1029-1041, doi: 10.3748/wjg.v26.i10.1029 (2020).
46 Lindahl, M. et al. Cell reports 7, 366-375, doi: 10.1016/j.celrep.2014.03.023 (2014).
47 Herranen, A. et al. Cell death & disease 11, 100, doi: 10.1038/s41419-020-2286-6 (2020).
48 Han, D. et al. Journal of drug targeting 30, 430-441, doi: 10.1080/1061186X.2021.2003803 (2022).
49 Yang, F. et al World journal of stem cells 12, 633-658, doi: 10.4252/wjsc.v12.i7.633 (2020).
50 Zhang, J. X. et al. Experimental gerontology 171, 112037, doi: 10.1016/j.exger.2022.112037 (2023).
51 Pakarinen, E. et al. eNeuro 7, doi: 10.1523/ENEURO.0477-19.2019 (2020).
52 Pakarinen, E. et al CMLS 79, 124, doi: 10.1007/s00018-022-04157-w (2022).
53 Roy, A. et al. eLife 10, doi: 10.7554/eLife.73215 (2021).26
54 Chen, L. et al. Scientific reports 5, 8133, doi: 10.1038/srep08133 (2015).
55 Oh, J. et al Aging 8, 2871-2896, doi: 10.18632/aging. 101098 (2016).
56 Yagi, T. et al. iScience 23, 101810, doi: 10.1016/j.isci.2020.101810 (2020).
57 Ren, H. et al. Molecular immunology 150, 78-89, doi: 10.1016/j.molimm.2022.08.003 (2022).
58 Sereno, D. et al Biochemistry and biophysics reports 11, 161-173, doi: 10.1016/j.bbrep.2017.02.009 (2017).
59 Doyle, S. E. et al. The Journal of experimental medicine 199, 81-90, doi: 10.1084/jem.20031237 (2004).
60 Ortuste Quiroga, H. P. et al Methods Mol Biol 1460, 85-100, doi: 10.1007/978-1-4939-3810-0_8 (2016).
61 Bencze, M., et al. Journal of visualized experiments: JoVE, doi: 10.3791/59754 (2019).
62 Galli, E. et al Scientific reports 9, 14318, doi: 10.1038/s41598-019-50841-6 (2019).

1. A method of increasing the regeneration of a skeletal muscle in an individual in need thereof comprising;
increasing the concentration of Mesencephalic Astrocyte-derived Neurotrophic Factor (MANF) in the skeletal muscle.

2. A method according to claim 1 wherein an increased concentration of MANF in the skeletal muscle increases the regeneration of the skeletal muscle.

3. A method according to claim 1 wherein the concentration of MANF is increased by administering to the individual an agent that increases the concentration of MANF in the skeletal muscle.

4. A method according to claim 3 wherein the agent is MANF.

5. A method according to claim 4 wherein MANF comprises the amino acid sequence of residues 25 to 182 of SEQ ID NO: 1 or a variant thereof.

6. A method according to claim 3 wherein the agent is a nucleic acid that encodes MANF.

7. A method according to claim 6 wherein the nucleic acid comprises the nucleotide sequence of SEQ ID NO: 2 or a variant thereof.

8. A method according to claim 7 wherein the nucleic acid is operably linked to a regulatory element.

9. A method according to claim 6 wherein the nucleic acid is contained in a vector.

10. A method according to claim 9 wherein the vector is a viral vector.

11. A method according to claim 3 wherein the agent is a cell that expresses MANF.

12. A method according to claim 11 wherein the cell comprises a heterologous nucleic acid comprising the nucleotide sequence of SEQ ID NO: 2 or a variant thereof.

13. A method according to claim 3 wherein the agent is administered intra-muscularly.

14. A method according claim 1 wherein the individual has reduced capacity for skeletal muscle regeneration.

15. A method according to claim 14 wherein the individual has a defect in skeletal muscle regeneration.

16. A method according to claim 15 wherein the defect is an age-related defect.

17. A method according to claim 16 wherein the individual is a human of greater than 60 years old.

18. A method according to claim 1 wherein the individual has skeletal muscle damage.

19. A method according to claim 1 wherein the individual has a skeletal muscle disease.

20. A method according to claim 19 wherein the muscle disease is a muscular dystrophy.

21. A method according to claim 20 wherein the muscular dystrophy is Duchenne Muscle Dystrophy.

22. A method according to claim 19 wherein the muscle disease is a neuromuscular disease.

23. A method according to claim 22 wherein the disease is spinal muscular atrophy, peripheral nerve disease, amyotrophic lateral sclerosis (ALS) or neuromuscular junction disease.

24. A method according to claim 19 wherein the muscle disease is a myopathy.

25. A method according to claim 24 wherein the myopathy is inflammatory myopathy, dermatomyositis, mitochondrial myopathy or metabolic myopathy.

26-29. (canceled)


SEQUENCE LISTING (2 sequences)

SEQ ID NO: 1 - protein, 182 aa, Homo sapiens
MRRMWATQGLAVALALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASARTDL

SEQ ID NO: 2 - RNA, 909 nt, Homo sapiens
  source 1..909: mol_type = genomic RNA
agtcggtcggcggcggcagcggaggaggaggaggaggaggaggatgaggaggatgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgtagtctgctcaatctctgttgcacctgagggggaaaaaacagttcaactgcttactcccaaaacagcctttttgtaatttattttttaagtgggctcctgacaatactgtatcagatgtgaagcctggagctttcctgatgatgctggccctacagtacccccatgaggggattcccttccttctgttgctggtgtactctaggacttcaaagtgtgtctgggatttttttattaaagaaaaaaaatttctagctgtccttgcagaattatagtgaataccaaaatggggttttgccccaggaggctccta